(19) United States
(12) Patent Application Publication
(10) Pub. No.: US 2007/0212306 A1
(43) Pub. Date: Sep. 13, 2007

(54) INTRANASAL FORMULATIONS OF INTERFERON BETA FREE OF STABILIZERS THAT ARE PROTEINS OR POLYPEPTIDES

(76) Inventors: Steven C. Quay, Seattle, WA (US); Henry R. Costantino, Woodinville, WA (US)

Correspondence Address: NASTECH PHARMACEUTICAL COMPANY INC, 3830 MONTE VILLA PARKWAY, BOTHELL, WA, 98021-7266, US

(21) Appl. No.: 11/570,068

(22) PCT Filed: Jun. 7, 2005
(86) PCT No.: PCT/US05/19908

§ 371 Date: Dec. 5, 2006

Related U.S. Application Data
(60) Provisional application No. 60/577,695, filed on Jun. 7, 2004.

(51) Int. Cl. A61K 9/12; A61K 38/21

(52) U.S. Cl. 424/45; 424/85.5

(57) ABSTRACT

Compositions and methods are provided for intranasal delivery of interferon-β yielding improved pharmacokinetic and pharmacodynamic results wherein the composition is free of a stabilizer that is a protein or a polypeptide. In certain aspects of the invention, the interferon-β is delivered to the intranasal mucosa along with one or more intranasal delivery-enhancing agent(s) to yield substantially increased absorption and/or bioavailability of the interferon-β and/or a substantially decreased time to maximal concentration of interferon-β in a tissue of a subject as compared to controls where the interferon-β is administered to the same intranasal site alone or formulated according to previously disclosed reports. The enhancement of intranasal delivery of interferon-β according to the methods and compositions of the present invention allows for the effective pharmaceutical use of these agents to treat a variety of diseases and conditions in mammalian subjects.

A major disadvantage of drug administration by injection is that trained personnel are often required to administer the drug. For self-administered drugs, many patients are reluctant or unable to give themselves injections on a regular basis. Injection is also associated with increased risks of infection. Some drugs, like beta interferon, can cause tissue necrosis when injected subcutaneously or even intramuscularly to the point of requiring surgical debridement of the wounds created. Other disadvantages of drug injection include variability of delivery results between individuals, as well as unpredictable intensity and duration of drug action.

Mucosal administration of therapeutic compounds may offer certain advantages over injection and other modes of administration, for example in terms of convenience and speed of delivery, as well as by reducing or eliminating compliance problems and side effects that attend delivery by injection. However, mucosal delivery of interferon betas is limited by mucosal barrier functions and other factors. For these reasons, mucosal drug administration typically requires larger amounts of drug than administration by injection. Other therapeutic compounds, including large molecule drugs, peptides and proteins, are often refractory to mucosal delivery.

One group of therapeutic compounds of interest for mucosal delivery is interferon-β IFN-β exhibits a potent antiviral function. IFN-β also mediates a variety of immunoregulatory effects.

Interferon β has been reported for treatment of relapsing forms of multiple sclerosis (MS). MS is a chronic, often disabling disease of the central nervous system. It is caused by the autoimmune destruction of myelin. Myelin is the fatty tissue that surrounds and protects central nervous system nerve fibers and facilitates the flow of nerve impulses to and from the brain. The loss of myelin disrupts the conduction of nerve impulses, producing the symptoms of MS. Symptoms may be mild numbness in the limbs, or severe paralysis or loss of vision.

IFN-β, alone or in combination with IFN-α has also been reported for treating active or chronic hepatitis B. IFN-β can be used for treatment and prevention of condyloma acuminata (genital or venereal warts caused by papilloma virus infection), papillomavirus warts of the larynx and skin (common warts). The antiviral activity of IFN-β is also reported to be useful in the treatment of severe childhood viral encephalitis.

Three forms of IFN-β approved for treatment of multiple sclerosis (MS) in the United States are IFN-β-1a (Avonex®, Biogen, Inc and Rebif®: Serono, Inc.) and IFN-β-1b (Betaseron®, Berlex Laboratories). IFN-β-1a differs from IFN-β-1b in several respects. IFN-β-1a is generated in mammalian cell culture (Chinese hamster ovary cells) whereas IFN-β-1b is produced in bacterial cells (Escherichia coli). IFN-β-1a amino acid sequence is identical to naturally occurring interferon. IFN-β-1b amino acid sequence substitutes serine for cysteine at position 17 of the 165-amino acid interferon protein.

Previous formulations of intranasal interferon beta have contained stabilizers that are proteins or polypeptides such as human serum albumin or bovine serum albumin. However, such proteins add an additional expense to the formulation and run the risk of contamination by viral agents such as hepatitis, or prion agents such as bovine spongiform encephalopathy. Thus, there is a need to produce a stable intranasal formulation of interferon beta that is free of stabilizers that are proteins or polypeptides, such as human or bovine serum albumin.

### DESCRIPTION OF THE INVENTION

The present invention fulfills the foregoing needs and satisfies additional objects and advantages by providing novel, effective methods and-compositions for intranasal delivery of interferon-β, which are free of stabilizers that are proteins or polypeptides yielding improved pharmacokinetic and pharmacodynamic results. In certain aspects of the invention, the interferon-β is delivered to the intranasal mucosa along with one or more intranasal delivery-enhancing agent(s) to yield substantially increased absorption and/or bioavailability of the interferon-β and/or a substantially decreased time to maximal concentration of interferon-β in a tissue of a subject as compared to controls where the interferon-β is administered to the same intranasal site alone or formulated according to previously disclosed reports. The enhancement of intranasal delivery of interferon-β according to the methods and compositions of the present invention allows for the effective pharmaceutical use of these agents to treat a variety of diseases and conditions in mammalian subjects.

The methods and compositions provided herein provide for enhanced delivery of interferon-β across nasal mucosal barriers to reach novel target sites for drug action yielding an enhanced, therapeutically effective rate or concentration of delivery. In certain aspects, employment of one or more intranasal delivery-enhancing agents facilitates the effective delivery of interferon-β to a targeted, extracellular or cellular compartment, for example the systemic circulation, a selected cell population, tissue or organ. Exemplary targets for enhanced delivery in this context are target physiological compartments, tissues, organs and fluids (e.g., within the blood serum, central nervous system (CNS) or cerebral spinal fluid (CSF)) or selected tissues or cells of the liver, bone, muscle, cartilage, pituitary, hypothalamus, kidney, lung, heart, testes, skin, or peripheral nervous system.

The enhanced delivery methods and compositions of the present invention provide for therapeutically effective mucosal delivery of interferon-β for prevention or treatment of a variety of diseases and conditions in mammalian subjects. Interferon-β can be administered via a variety of mucosal routes, for example by contacting interferon-β to a nasal mucosal epithelium, a bronchial or pulmonary mucosal epithelium, an oral, gastric, intestinal or rectal mucosal epithelium, or a vaginal mucosal epithelium. Typically, the methods and compositions are directed to or formulated for intranasal delivery (e.g., nasal mucosal delivery or intranasal mucosal delivery).

In one aspect of the invention, pharmaceutical formulations suitable for intranasal administration are provided that comprise a therapeutically effective amount of interferon-β and one or more intranasal delivery-enhancing agents as described herein, which formulations are effective in a nasal mucosal delivery method of the invention to prevent the onset or progression of disease related to autoimmune disease, viral infection, or cancer, e.g., a solid tumor, in a mammalian subject, or to alleviate one or more clinically recognized symptoms of autoimmune disease, viral infection or cancer in a mammalian subject.

In another aspect of the invention, pharmaceutical formulations suitable for intranasal administration are provided that comprise a therapeutically effective amount of interferon-β and one or more intranasal delivery-enhancing agents as described herein, which formulation is effective in a nasal mucosal delivery method of the invention to alleviate symptoms or prevent the onset or lower the incidence or severity of multiple sclerosis, condyloma acuminata (genital or venereal warts caused by papilloma virus infection), papillomavirus warts of the larynx and skin (common warts), chronic hepatitis B, or severe childhood viral encephalitis. In more detailed aspects of the invention, methods and compositions for intranasal delivery of interferon-β incorporate one or more intranasal delivery enhancing agent(s) combined in a pharmaceutical formulation together with, or administered in a coordinate nasal mucosal delivery protocol with, a therapeutically effective amount of IFN-\tilde{β}. These methods and compositions provide enhanced nasal transmucosal delivery of the interferon-β, often in a pulsatile delivery mode to maintain continued release of interferon-β to yield more consistent (normalized) or elevated therapeutic levels of interferon-β in the blood serum, central nervous system (CNS), cerebral spinal fluid (CSF), or in another selected physiological compartment or target tissue or organ for treatment of disease. Normalized and elevated therapeutic levels of interferon-β are determined, for example, by an increase in bioavailability (e.g., as measured by maximal concentration (Cₘₐₓ) or the area under concentration vs. time curve (AUC) for an intranasal effective amount of interferon-β) and/or an increase in delivery rate (e.g., as measured by time to maximal concentration (tₘₐₓ), Cₘₐₓ, and or AUC). Normalized and elevated high therapeutic levels of interferon-β in the blood serum, central nervous system (CNS), or cerebral spinal fluid (CSF) may be achieved in part by repeated intranasal administration to a subject within a selected dosage period, for example an 8, 12, or 24 hour dosage period.

To maintain more consistent or normalized therapeutic levels of interferon-β, the pharmaceutical formulations of the present invention are often repeatedly administered to the nasal mucosa of the subject, for example one, two or more times within a 24 hour period, four or more times within a 24 hour period, six or more times within a 24 hour period, or eight or more times within a 24 hour period. The methods and compositions of the present invention yield improved pulsatile delivery to maintain normalized and/or elevated therapeutic levels of interferon-β e.g., in the blood serum. The methods and compositions of the invention enhance transnasal mucosal delivery of interferon-β to a selected target tissue or compartment by at least a two- to five-fold increase, more typically a five- to ten-fold increase, and commonly a ten- to twenty-five- up to a fifty-fold increase (e.g., as measured by tₘₐₓ Cₘₐₓ, and/or AUC, in the blood serum, central nervous system, cerebral spinal fluid, or in another selected physiological compartment or target tissue or organ for delivery), compared to the efficacy of delivery of interferon-β administered alone or using a previously-described delivery method, for example a previously-described mucosal delivery, intramuscular delivery, subcutaneous delivery, intravenous delivery, and/or parenteral delivery method.

Nasal mucosal delivery of interferon-β according to the methods and compositions of the invention will often yield effective delivery and bioavailability that approximates dosing achieved by continuous administration methods. In other aspects, the invention provides enhanced nasal mucosal delivery that permits the use of a lower systemic dosage and significantly reduces the incidence of interferon-\tilde{β}related side effects. Because continuous infusion of interferon-β outside the hospital setting is otherwise impractical, mucosal delivery of interferon-β as provided herein yields unexpected advantages that allow sustained delivery of interferon-β, with the accrued benefits, for example, of improved patient-to-patient dose variability.

In more detailed aspects of the invention, the methods and compositions of the present invention provide improved and/or sustained delivery of interferon-β to the blood serum, lymphatic system, CNS, and/or CSF. In one exemplary embodiment, an intranasal effective amount of interferon-β and one or more intranasal delivery enhancing agent(s) is contacted with a nasal mucosal surface of a subject to yield enhanced mucosal delivery of interferon-β to the central nervous system (CNS) or cerebral spinal fluid (CSF) of the subject, for example to effectively treat autoimmune diseases. In certain embodiments, the methods and compositions of the invention provide improved and sustained delivery of interferon-β to the CNS and will effectively treat one or more symptoms of multiple sclerosis, including in cases where conventional interferon-β therapy yields poor results or unacceptable adverse side effects.

In exemplary embodiments, the methods and compositions of the present invention yield a two- to five-fold decrease, more typically a five- to ten-fold decrease, and commonly a ten- to twenty-five- up to a fifty- to one hundred-fold decrease in the time to maximal concentration (tₘₐₓ) of the interferon-β in blood serum, central nervous system, cerebral spinal fluid, and/or in another selected physiological compartment or target tissue or organ for delivery—as compared to delivery rates for interferon-β administered alone or in accordance with previously-described drug delivery methods.

In further exemplary embodiments, the methods and compositions of the invention yield a two- to five-fold increase, more typically a five- to ten-fold increase, and commonly a ten- to twenty-five- up to a fifty- to one hundred-fold increase in the area under concentration vs. time curve, AUC, of the interferon-β in blood serum, central nervous system, cerebral spinal fluid, and/or in another selected physiological compartment or target tissue or organ for delivery—as compared to delivery rates for the interferon-β administered alone or in accordance with previously-described administration methods.

In further exemplary embodiments, the methods and compositions of the present invention yield a two- to five-fold increase, more typically a five- to ten-fold increase, and commonly a ten- to twenty-five- up to a fifty- to one hundred-fold increase in the maximal concentration, Cₘₐₓ, of the interferon-β in blood serum, central nervous system, cerebral spinal fluid, and/or in another selected physiological compartment or target tissue or organ for delivery—as compared to delivery rates for the interferon-β administered alone or in accordance with previously-described administration methods.

The methods and compositions of the invention will often serve to improve interferon-β dosing schedules and thereby maintain normalized and/or elevated, therapeutic levels of interferon-β in the subject. In certain embodiments, the invention provides compositions and methods for intranasal delivery of interferon-β, wherein interferon-β dosage normalized and sustained by repeated, typically pulsatile, delivery to maintain more consistent, and in some cases elevated, therapeutic levels. In exemplary embodiments, the time to maximum concentration (tₘₐₓ) of interferon-β in the blood serum will be from about 0.1 to 4.0 hours, alternatively from about 0.4 to 1.5 hours, and in other embodiments from about 0.7 to 1.5 hours, or from about 1.0 to 1.3 hours. Thus, repeated intranasal dosing with the formulations of the invention, on a schedule ranging from about 0.1 to 2.0 hours between doses, will maintain normalized, sustained therapeutic levels of interferon-β to maximize clinical benefits while minimizing the risks of excessive exposure and side effects.

In alternative embodiments, the invention achieves enhanced delivery of normalized and/or elevated, improved therapeutic levels of interferon-β by combining mucosal administration of one dosage amount of interferon-β formulated with one or more intranasal delivery-enhancing agents, with a separate dosage amount of interferon-β delivered by a non-mucosal route, for example by intramuscular administration. In one exemplary embodiment, intranasal delivery of interferon-β according to the compositions and methods herein yields normalized and/or elevated, high therapeutic levels of interferon-β in the blood serum of the subject for a time period between approximately 0.1 and 3 hours following intranasal administration. Coordinate administration of interferon-β by a non-mucosal route (before, simultaneous with, or after mucosal administration) provides more consistent, elevated therapeutic levels of interferon-β in the blood serum of the subject for an effective time period of between approximately 2 to 24 hours, more often between about 4-16 hours, and in certain embodiments between about 6-8 hours. Within these coordinate administration methods, improving clinical benefit while minimizing the risks of excessive exposure facilitates the aims of the treating physician.

In other aspects of the invention, the methods and formulations for intranasally administering interferon-β described herein yield a significantly enhanced rate or level of delivery (e.g., decreased tₘₐₓ, increased AUC, and/or increased Cₘₐₓ) of the interferon-β into the serum, or to selected tissues or cells, of the subject. This includes enhanced delivery rates or levels into the serum, or to selected tissues or cells (e.g., blood serum, CNS, or CSF), compared to delivery rates and levels for the interferon-β administered alone or in accordance with previously-described technologies. Thus, in certain aspects of the invention, the foregoing methods and compositions are administered to a mammalian subject to yield enhanced delivery of the interferon-β to a physiological compartment, fluid, tissue or cell within the mammalian subject.

Within more detailed aspects of the invention, bioavailability of interferon-β achieved by the methods and formulations herein (e.g., measured by peak blood plasma levels (Cₘₐₓ) of interferon-β in blood serum, CNS, CSF or in another selected physiological compartment or target tissue) will be, for example, about 5 μg per liter of blood plasma or CSF, typically about 10 μg per liter of blood plasma or CSF, about 20 μg per liter of blood plasma or CSF, about 30 μg per liter of blood plasma or CSF, about 40 μg per liter of blood plasma or CSF, about 50 μg per liter of blood plasma or CSF, or about 60 μg or greater per liter of blood plasma or CSF.

Within other detailed aspects of the invention, bioavailability of interferon-β following administration in accordance with the methods and compositions of the invention is determined by measuring interferon-β “pharmacokinetic markers”. For example, art-accepted pharmacokinetic markers for interferon-β, serum β-2 microglobulin or serum neopterin, may be measured following administration, e.g., as measured by peak blood plasma levels (Cₘₐₓ) of the marker(s) in blood serum, CNS, CSF or in another selected physiological compartment or target tissue. These and other such marker data are accepted in the art as reasonably correlated with pharmakokinetics of interferon-β compounds that may be undetectable directly in vivo. In certain aspects, enhanced bioavailability of interferon-β as measured by interferon-β markers will be demonstrated by, for example, a correlated Cₘₐₓ for serum β-2 microglobulin of approximately 1.7 mg/ml of blood plasma or CSF, or approximately 2.0 mg/ml of blood plasma or CSF, or approximately 4.0 mg/ml or greater of blood plasma or CSF. Cₘₐₓ for serum neopterin of approximately 8 nmol/l of blood plasma or CSF, approximately 10 nmol/l of blood plasma or CSF, approximately 20 nmol/l of blood plasma or CSF, approximately 30 nmol/l of blood plasma or CSF, or approximately 40 nmol/l or greater of blood plasma or CSF.

Within further detailed aspects, the pharmaceutical composition as disclosed herein following mucosal administration to said subject yields a peak concentration (Cₘₐₓ) for pharmacological markers, neopterin or β2-microglobulin in the blood plasma or CNS tissue or fluid of the subject that is typically 25% or greater, or 75% or greater, or 150% or greater, as compared to a peak concentration of neopterin or β2-microglobulin in blood plasma or CNS tissue or fluid following intramuscular injection of an equivalent concentration or dose of interferon-β to said subject, intranasal delivery of interferon-β alone, and/or mucosal delivery of interferon-β using previously-described methods and formulations.

Within other detailed aspects of the invention, bioavailability of interferon-β as will be determined by measuring interferon-β pharmacokinetic markers, for example, serum β-2 microglobulin or serum neopterin, to determine area under the concentration curve (AUC) for the marker(s) in blood serum, CNS, CSF or in another selected physiological compartment or target tissue. Bioavailability of interferon-β as determined by interferon-β markers in this context will be, for example, AUC_{0-96 hr} for serum β-2 microglobulin of approximately 200 μIU•hr/mL of blood plasma or CSF, AUC_{0-96 hr} for β-2 microglobulin up to approximately 500 μIU•hr/mL of blood plasma or CSF, AUC_{0-96 hr} for serum neopterin of approximately 200 ng·hr/ml of blood plasma or CSF, AUC_{0-96 hr} for serum neopterin up to approximately 500 ng·hr/ml of blood plasma or CSF.

Within further detailed aspects, the pharmaceutical composition as disclosed herein following mucosal administration to said subject yields area under the concentration curve (AUC_{0-96 hr}) for pharmacological markers, neopterin or β2-microglobulin, in the blood plasma or CNS tissue or fluid of the subject that is typically 25% or greater, or 75% or greater, or 150% or greater, as compared to an AUC_{0-96 hr} for neopterin or β2-microglobulin in blood plasma or CNS tissue or fluid following intramuscular injection of an equivalent concentration or dose of interferon-β to said subject, intranasal delivery of interferon-β alone, and/or mucosal delivery of interferon-β using previously-described methods and formulations.

Within yet additional detailed aspects of the invention, bioavailability of interferon-β pharmacokinetic markers, for example, serum β-2 microglobulin or serum neopterin, achieved by the methods and formulations herein is measured by time to maximal concentration (tₘₐₓ) in blood serum, CNS, CSF or in another selected physiological compartment or target tissue. tₘₐₓ for serum β-2 microglobulin will be, for example, between about 45 hours or less and about 48 to 60 hours. In other embodiments, these values may be 35 hours or less, or 25 hours or less following intranasal administration of interferon-β by methods and formulations described herein. tₘₐₓ for serum neopterin will be, for example, about 40 hours or less, typically 30 hours or less, or typically 25 hours or less following intranasal administration of interferon-β by methods and formulations described herein.

Within further detailed aspects, the pharmaceutical composition as disclosed herein following mucosal administration to said subject yields a time to maximal plasma concentration (tₘₐₓ) for pharmacological markers, neopterin or β₂-microglobulin, in a blood plasma or CNS tissue or fluid of the subject that is typically between about 25 to 45 hours, or between about 25 to 35 hours.

In exemplary embodiments, administration of one or more interferon-β formulated with one or more intranasal delivery-enhancing agents as described herein yields effective delivery to the blood serum CNS, or CSF to alleviate a selected disease or condition (e.g., multiple sclerosis, or a symptom thereof) in a mammalian subject. In more detailed aspects, the methods and formulations for intranasally administering interferon-β according to the invention yield a significantly enhanced rate or level of delivery (e.g., decreased tₘₐₓ or increased Cₘₐₓ) of the interferon-β into the serum or to selected tissues or cells (e.g., liver), compared to delivery rates and levels for the interferon-β administered alone or in accordance with previously-described technologies.

Within exemplary aspects, the enhanced delivery rate or level of the interferon-β provides for more effective treatment of multiple sclerosis or viral disease in a subject. For example, by using the intranasal administration methods and formulations of the invention, an effective concentration of interferon-β can be delivered to the blood serum CNS, CSF, or peripheral nervous system, usually within about 45 min, 30 min, 20 min, and even 15 min or less following administration, resulting in an enhanced therapeutic effect (e.g., decreased symptoms of MS, or decreased viral load) in the subject with minimal side effects. Side effects that are generally minimized or avoided by the methods and compositions of the invention include progressive damage and bleeding to the mucosal site of drug delivery from repeated administration—that would otherwise result in poor mucosal absorption of interferon-β. Additional side effects that are reduced or avoided by the present invention include flu-like syndrome of headache, fever, malaise, sensations of temperature change myalgias, arthralgias, and severe delivery site reactions such as necrosis, nausea, leucopoenia, and liver enzyme abnormalities.

The enhanced pharmacokinetics of delivery of interferon-β (e.g., increased frequency of dosing possible, increased rate, normalized, sustained delivery, and elevated levels) according to the methods of the invention, provides improved therapeutic efficacy, e.g., to treat autoimmune disease, viral infection, or cancer in a subject, without unacceptable adverse side effects. Thus, for example, pharmaceutical preparations formulated for nasal mucosal delivery are provided for treating multiple sclerosis in a mammalian subject that comprise a therapeutic intranasal effective amount of interferon-β combined with one or more intranasal delivery-enhancing agents as disclosed herein. These preparations surprisingly yield enhanced mucosal absorption of the interferon-β to produce a therapeutic effective concentration of the drug (e.g., for treating acute MS, or relapsing remitting MS in a subject) at a target site or tissue in the subject in about 45 minutes or less, 30 minutes or less, 20 minutes or less, or as little as 15 minutes or less.

Within other detailed embodiments of the invention, the foregoing methods and formulations are administered to a mammalian subject to yield enhanced bioavailability, or enhanced blood plasma concentration of mucosally-administered interferon-β, a cumulative (e.g., ‘per week’) area under the concentration curve (AUC) for interferon-β (e.g., as expressed by the AUC of a single dose multipled by the number of doses per week) in the blood plasma or CSF following mucosal (e.g., intranasal) administration to the subject by methods and compositions of the present invention is about 10% or greater compared to an area under the concentration curve (AUC) for interferon-β in the plasma or CSF following intramuscular injection to the mammalian subject. In exemplary embodiments, an area under the concentration curve (AUC) for interferon-β in the blood plasma or CSF following intranasal administration of one or more interferon-βs formulated with one or more intranasal delivery-enhancing agents as described herein is at leaset about 25%, 40%, or greater compared to an area under the concentration curve (AUC) for interferon-β in the plasma or CSF following intramuscular injection to the mammalian subject. In yet additional exemplary embodiments an area under the concentration curve (AUC) for interferon-β in the blood plasma or CSF following intranasal administration by methods and compositions of the present invention to the subject is at least about 60%, 80%, 100% or greater, up to 150% or greater, compared to an area under the concentration curve (AUC) for interferon-β in the plasma or CSF following intramuscular injection to the mammalian subject. These enhanced rates and levels of delivery are correlated with increased therapeutic efficacy of the methods and formulations of the invention for prophylaxis and treatment of the indicated diseases and conditions in mammalian subjects as compared to relevant clinical control subjects.

Within other detailed embodiments of the invention, the foregoing methods and formulations are administered to a mammalian subject to yield enhanced blood plasma or CSF levels of interferon-β, wherein following mucosal (e.g., intranasal) administration of interferon-β according to the methods and compositions herein yields a time to maximal plasma or CSF concentration (tₘₐₓ) for interferon-β between approximately 0.1 to 4.0 hours. In exemplary embodiments a time to maximal plasma or CSF concentration (tₘₐₓ) of interferon-β in the blood plasma following intranasal administration by methods and compositions of the present invention to the subject is between approximately 0.7 to 1.5 hours, or between approximately 1.0 to 1.3 hours. In exemplary embodiments, a time to maximal plasma or CSF concentration (tₘₐₓ) of interferon-βpharmacokinetic markers, serum β-2 microglobulin or serum neopterin, following administration of one or more interferon-β formulated with one or more intranasal delivery-enhancing agents as described herein is between approximately 25 and 45 hours, or between approximately 25 to 30 hours. These enhanced rates and levels of delivery are correlated with increased therapeutic efficacy of the methods and formulations of the invention for prophylaxis and treatment of the indicated diseases and conditions in mammalian subjects as compared to relevant clinical control subjects.

Within other detailed embodiments of the invention, the foregoing methods and formulations are administered to a mammalian subject to yield enhanced blood plasma or CSF levels of interferon-β, whereby said formulation following mucosal (e.g., intranasal) administration to the subject yields a time to maximal plasma concentration (tₘₐₓ) of said interferon-β in a blood plasma or CSF of said subject that is 75%, 50%, 20%, or as short as 10% or less compared to a time to maximal plasma concentration (tₘₐₓ) of interferon-β in the blood plasma or CSF of the subject following administration of an equivalent concentration or dose of interferon-β by intramuscular injection. These enhanced rates and levels of delivery are correlated with increased therapeutic efficacy of the methods and formulations of the invention for prophylaxis and treatment of the indicated diseases and conditions in mammalian subjects as compared to relevant clinical control subjects.

Within other detailed embodiments of the invention, the foregoing methods and formulations are administered to a mammalian subject to yield enhanced blood plasma or CSF levels of mucosally-administered interferon-β, whereby a peak concentration of interferon-β in the blood plasma (Cₘₐₓ) following mucosal (e.g., intranasal) administration to the subject by methods and compositions of the present invention is about 25% or greater compared to a peak concentration of interferon-β in the plasma following intramuscular injection to the mammalian subject. In exemplary embodiments, a peak concentration of interferon-β in the blood plasma (Cₘₐₓ) following intranasal administration of interferon-β formulated with one or more intranasal delivery-enhancing agents as described herein is about 40% or greater compared to a peak concentration of interferon-β in the plasma following intramuscular injection to the mammalian subject. In yet additional exemplary embodiments a peak concentration of interferon-β in the blood plasma (Cₘₐₓ) following intranasal administration by methods and compositions of the present invention to the subject is about 80% or greater, about 100% or greater, up to 150% or greater, compared to a peak concentration of interferon-β in the plasma following intramuscular injection to the mammalian subject. These enhanced rates and levels of delivery are correlated with improved therapeutic efficacy of the methods and formulations of the invention for prophylaxis and treatment of the indicated diseases and conditions in mammalian subjects.

Within other detailed embodiments of the invention, the foregoing methods and formulations are administered to a mammalian subject to yield enhanced CNS, cerebral spinal fluid (CSF) or peripheral nervous system delivery of the interferon-β, whereby the peak interferon-β concentration in a CNS, CSF or peripheral nervous system target site by intranasal delivery (e.g., nasal mucosal delivery) is at least 5% of a related peak interferon-β concentration in the blood plasma following administration of the formulation to the subject. In exemplary embodiments, administration of one or more interferon-βs formulated with one or more intranasal delivery-enhancing agents as described herein yields a peak interferon-β concentration in the CNS, CSF, or peripheral nervous system of about 10% or greater versus the peak interferon-β concentration in the blood plasma following administration of the formulation to the subject. In other exemplary embodiments, the peak interferon-β concentration in the CNS, CSF or peripheral nervous system is about 15% or greater versus the peak interferon-β concentration in the blood plasma. In yet additional exemplary embodiments, the peak interferon-β concentration in the CNS, CSF or peripheral nervous system is about 20% or greater, 30% or greater, 35% or greater, or up to 40% or greater, versus the peak interferon-β concentration in the blood plasma. These enhanced rates and levels of delivery are correlated directly with the efficacy of the nasal mucosal delivery methods and formulations of the invention for prophylaxis and treatment of diseases and conditions in mammalian subjects amenable to prophylaxis and treatment by CNS, CSF or peripheral nervous system delivery of therapeutic levels of selected interferon-β.

Within other detailed embodiments of the invention, the foregoing methods and formulations are administered to a mammalian subject to yield enhanced blood plasma levels, CNS, CSF or other tissue levels of the interferon-β by administering a formulation comprising an intranasal effective amount of interferon-β and one or more intranasal delivery-enhancing agents and one or more sustained release-enhancing agents. The sustained release-enhancing agents, for example, may comprise a polymeric delivery vehicle. In exemplary embodiments, the sustained release-enhancing agent may comprise polyethylene glycol (PEG) coformulated or coordinately delivered with interferon-β and one or more intranasal delivery-enhancing agents. PEG may be covalently bound to interferon-β. The sustained release-enhancing methods and formulations of the present invention will increase residence time (RT) of the interferon-β at a site of administration and will maintain a basal level of the interferon-β over an extended period of time in blood plasma, CNS, CSF, or other tissue in the mammalian subject.

Within other detailed embodiments of the invention, the foregoing methods and formulations are administered to a mammalian subject to yield enhanced blood plasma levels, CNS, CSF or other tissue levels of the interferon-β to maintain basal levels of interferon-β over an extended period of time. Exemplary methods and formulations involve administering a pharmaceutical formulation comprising an intranasal effective amount of interferon-β and one or more intranasal delivery-enhancing agents to a mucosal surface of the subject, in combination with intramuscular administration of a second pharmaceutical formulation comprising interferon-β. Maintenance of basal levels of interferon-β is particularly useful for treatment and prevention of disease, for example, multiple sclerosis, papilloma virus infection, and chronic hepatitis B.

The foregoing mucosal drug delivery formulations and preparative and delivery methods of the invention provide improved mucosal delivery of interferon-β to mammalian subjects. These compositions and methods can involve combinatorial formulation or coordinate administration of one or more interferon-βs) with one or more mucosal (e.g., intranasal) delivery-enhancing agents. Among the mucosal delivery-enhancing agents to be selected from to achieve these formulations and methods are (a) aggregation inhibitory agents; (b) charge modifying agents; (c) pH control agents; (d) degradative enzyme inhibitors; (e) mucolytic or mucus clearing agents; (f) ciliostatic agents; (g) membrane penetration-enhancing agents (e.g., (i) a surfactant, (ii) a bile salt, (ii) a phospholipid or fatty acid additive, mixed micelle, liposome, or carrier, (iii) an alcohol, (iv) an enamine, (v) an NO donor compound, (vi) a long-chain amphipathic molecule (vii) a small hydrophobic penetration enhancer; (viii) sodium or a salicylic acid derivative; (ix) a glycerol ester of acetoacetic acid (x) a clyclodextrin or beta-cyclodextrin derivative, (xi) a medium-chain fatty acid, (xii) a chelating agent, (xiii) an amino acid or salt thereof, (xiv) an N-acetylamino acid or salt thereof, (xv) an enzyme degradative to a selected membrane component, (ix) an inhibitor of fatty acid synthesis, (x) an inhibitor of cholesterol synthesis; or (xi) any combination of the membrane penetration enhancing agents-of (i)-(x)); (h) modulatory agents of epithelial junction physiology, such as nitric oxide (NO) stimulators, chitosan, and chitosan derivatives; (i) vasodilator agents; (j) selective transport-enhancing agents; and (k) stabilizing delivery vehicles, carriers, supports or complex-forming species with which the interferon-βs is/are effectively combined, associated, contained, encapsulated or bound to stabilize the active agent for enhanced nasal mucosal delivery.

In various embodiments of the invention, interferon-β is combined with one, two, three, four or more of the mucosal (e.g., intranasal) delivery-enhancing agents recited in (a)-(k), above. These mucosal delivery-enhancing agents may be admixed, alone or together, with the interferon-β, or otherwise combined therewith in a pharmaceutically acceptable formulation or delivery vehicle. Formulation of interferon-β with one or more of the mucosal delivery-enhancing agents according to the teachings herein (optionally including any combination of two or more mucosal delivery-enhancing agents selected from (a)-(k) above) provides for increased bioavailability of the interferon-β following delivery thereof to a mucosal (e.g., nasal mucosal) surface of a mammalian subject.

In related aspects of the invention, a variety of coordinate administration methods are provided for enhanced mucosal delivery of interferon-β. These methods comprise the step, or steps, of administering to a mammalian subject a mucosally effective amount of at least one interferon-β in a coordinate administration protocol with one or more mucosal delivery-enhancing agents of (a)-(k) above.

To practice a coordinate administration method according to the invention, any combination of one, two or more of the mucosal delivery-enhancing agents recited in (a)-(k), above, may be admixed or otherwise combined for simultaneous mucosal (e.g., intranasal) administration. Alternatively, any combination of one, two or more of the mucosal delivery-enhancing agents recited in (a)-(k) can be mucosally administered, collectively or individually, in a predetermined temporal sequence separated from mucosal administration of the interferon-β (e.g., by pre-administering one or more of the delivery-enhancing agent(s)), and via the same or different delivery route as the interferon-β (e.g., to the same or to a different mucosal surface as the interferon-β, or even via a non-mucosal (e.g., intramuscular, subcutaneous, or intravenous route). Coordinate administration of interferon-β with any one, two or more of the mucosal delivery-enhancing agents according to the teachings herein provides for increased bioavailability of the interferon-β following delivery thereof to a mucosal surface of a mammalian subject.

In additional related aspects of the invention, various “multi-processing” or “co-processing” methods are provided for preparing formulations of interferon-β for enhanced nasal mucosal delivery. These methods comprise one or more processing or formulation steps wherein one or more interferon-β(s) is/are serially, or simultaneously, contacted with, reacted with, or formulated with, one, two or more (including any combination of) of the mucosal delivery-enhancing agents of (a)-(k) above.

To practice the multi-processing or co-processing methods according to the invention, the interferon-β is/are exposed to, reacted with, or combinatorially formulated with any combination of one, two or more of the mucosal delivery-enhancing agents recited in (a)-(k), above, either in a series of processing or formulation steps, or in a simultaneous formulation procedure, that modifies the interferon-β (or other formulation ingredient) in one or more structural or functional aspects, or otherwise enhances mucosal delivery of the active agent in one or more (including multiple, independent) aspect(s) that are each attributed, at least in part, to the contact, modifying action, or presence in a combinatorial formulation, of a specific mucosal delivery-enhancing agent recited in (a)-(k), above.

In certain detailed aspects of the invention, the methods and compositions which comprise a mucosally effective amount of interferon-β and one or more mucosal delivery-enhancing agent(s) (combined in a pharmaceutical formulation together or administered in a coordinate nasal mucosal delivery protocol) provide nasal transmucosal delivery of the interferon-β in a pulsatile delivery mode to maintain more consistent or normalized, and/or elevated levels of interferon-β in the blood serum. In this context, the pulsatile delivery methods and compositions of the invention yield increased bioavailability (e.g., as measured by maximal concentration, (Cₘₐₓ) or area under concentration curve (AUC) of interferon-β and/or an increased mucosal delivery rate (e.g., as measured by time to maximal concentration (tₘₐₓ), Cₘₐₓ and/or AUC compared to other mucosal or non-mucosal delivery method-based controls. For example, the invention provides pulsatile delivery methods and formulations that comprise interferon-β and one or more mucosal delivery-enhancing agent(s), wherein the formulation administered mucosally (e.g., intranasally) to a mammalian subject, yields an area under the concentration curve (AUC) for interferon-β in the blood plasma that is about 10% or greater compared to an area under the concentration curve (AUC) for interferon-β in the plasma following intramuscular injection to the mammalian subject.

Often the formulations of the invention are administered to a nasal mucosal surface of the subject. In certain embodiments, the interferon-β is a human interferon-β-1a, (Avonex®, Biogen, Inc.), human interferon-β-1b (Betaseron®, Berlex Laboratories), or a pharmaceutically acceptable salt or derivative thereof. A mucosally effective dose within the pharmaceutical formulations of the present invention comprises, for example, between about 10 μg and 600 μg of interferon-β. In certain embodiments, an effective dose of the pharmaceutical formulation comprising interferon-β is, for example, 30 μg, 60 μg, 90 μg, 120 μg, 200 μg, 250 μg, 300 μg, or 400 μg. In certain embodiments, an effective dose within the pharmaceutical formulations of the invention is, for example, between about 30 and 100 μg of interferon-β. The pharmaceutical formulations of the present invention may be administered in a repeated dosing regimen, for example, one or more times daily, 3 times per week, or weekly. In certain embodiments, the pharmaceutical formulations of the invention are administered two times daily, four times daily, or six times daily. In related embodiments, the mucosal (e.g., intranasal) formulations comprising interferon-β(s) and one or more delivery-enhancing agent(s) administered via a repeated dosing regimen yields an area under the concentration curve (AUC) for interferon-β in the blood plasma or CSF following repeated dosing that is about 25% or greater compared to an area under the concentration curve (AUC) for interferon-β in the plasma or CSF following one or more intramuscular injections of the same or comparable amount of interferon-β. In other embodiments, the mucosal formulations of the invention administered via a repeated dosing regimen yields an area under the concentration curve (AUC) for interferon-β in the blood plasma or CSF following repeated dosing that is about 40%, 80%, 100%, 150%, or greater compared to the AUC for interferon-β in the plasma 25% or greater compared to an area under the concentration curve (AUC) for interferon-β in the plasma or CSF following one or more intramuscular injections of the same or comparable amount of interferon-β.

In certain detailed aspects of the invention, a stable pharmaceutical formulation is provided which comprises interferon-β and one or more delivery-enhancing agent(s), wherein the formulation administered intranasally to a mammalian subject yields a time to maximal plasma concentration (tₘₐₓ) for interferon-β between approximately 0.4 to 2.0 hours in a mammalian subject. Often the formulation is administered to a nasal mucosal surface of the subject.

In certain embodiments of the invention, the intranasal formulation of interferon-β and one or more delivery-enhancing agent(s) yields a time to maximal plasma concentration (tₘₐₓ) for interferon-β between approximately 0.4 to 1.5 hours in the mammalian subject. Alternately, the intranasal formulation of the present invention yields a time to maximal plasma concentration (tₘₐₓ) for interferon-β between approximately 0.7 to 1.5 hours, or between approximately 1.0 to 1.3 hours in the mammalian subject.

In certain detailed aspects of the invention, a stable pharmaceutical formulation is provided which comprises interferon-β and one or more intranasal delivery-enhancing agent(s), wherein the formulation administered intranasally to a mammalian subject yields a peak concentration of interferon-β in the blood plasma (Cₘₐₓ) following intranasal administration to the subject by methods and compositions of the present invention that is about 25% or greater compared to a peak concentration of interferon-β in the plasma following intramuscular injection to the mammalian subject. Within related methods, the formulation is administered to a nasal mucosal surface of the subject.

In other detailed embodiments of the invention, the intranasal formulation of the interferon-β(s) and one or more delivery-enhancing agent(s) yields a peak concentration of interferon-β in the blood plasma (Cₘₐₓ) following intranasal administration to the subject that is about 40% or greater compared to a peak concentration of interferon-β in the plasma following intramuscular injection of a comparable dose of interferon-β to the subject. Alternately, the intranasal formulation of the present invention may yield a peak concentration of interferon-β in the blood plasma (Cₘₐₓ) that is about 80%, 100% or 150%, or greater compared to the peak concentration of interferon-β in the plasma following intramuscular injection to the mammalian subject.

Intranasal delivery-enhancing agents are employed which enhance delivery of interferon-β into or across a nasal mucosal surface. For passively absorbed drugs, the relative contribution of paracellular and transcellular pathways to drug transport depends upon the pKₐ, partition coefficient, molecular radius and charge of the drug, the pH of the luminal environment in which the drug is delivered, and the area of the absorbing surface. The intranasal delivery-enhancing agent of the present invention may be a pH control agent. The pH of the pharmaceutical formulation of the present invention is a factor affecting absorption of interferon-β via paracellular and transcellular pathways to drug transport. In one embodiment, the pharmaceutical formulation of the present invention is pH adjusted to between about pH 3.0 to 8.0. In a further embodiment, the pharmaceutical formulation of the present invention is pH adjusted to between about pH 3.5 to 7.5. In a further embodiment, the pharmaceutical formulation of the present invention is pH adjusted to between about pH 4.0 to 5.0. In a further embodiment, the pharmaceutical formulation of the present invention is pH adjusted to between about pH 4.0 to 4.5.

As noted above, the present invention provides improved methods and compositions for mucosal delivery of interferon-β (IFN-β) to mammalian subjects for treatment or prevention of a variety of diseases and conditions. Examples of appropriate mammalian subjects for treatment and prophylaxis according to the methods of the invention include, but are not restricted to, humans and non-human primates, livestock species, such as horses, cattle, sheep, and goats, and research and domestic species, including dogs, cats, mice, rats, guinea pigs, and rabbits.

In order to provide better understanding of the present invention, the following definitions are provided:

Interferon-β: As used herein, “interferon-β” or “IFN-β” refers to interferon-β in native-sequence or in variant form, and from any source, whether natural, synthetic, or recombinant. Natural IFN-β is a glycoprotein (approximately 20 percent sugar moiety) of 20 kDa and has a length of 166 amino acids. Glycosylation is not required for biological activity in vitro. The protein contains a disulfide bond Cys31/141 required for biological activity. The human gene encoding IFN-β has a length of 777 bp and maps to chromosome 9q22 in the vicinity of the IFN-α gene cluster. The IFN-β gene does not contain introns. A single gene encodes the human IFN-β. At least three different genes have been found encoding bovine IFN-β. IFN-β is also known as: fibroblast interferon, Type 1 interferon, pH2-stable interferon, and R1-GI factor.

IFN-β includes, for example, human interferon-β (h IFN-β) that is a natural or recombinant IFN-β with the human native sequence. Recombinant interferon-β (rIFN-β) refers to any IFN-β or variant produced by means of recombinant DNA technology. Two subtypes of human IFN-β, IFN-β-1a (Avonex®, Biogen, Inc.) and IFN-β-1b (Betaseron®, Chiron Corp.), have been approved for the treatment and prevention of multiple sclerosis, and other diseases.

Additional disclosures teach detailed methods and tools pointing to specific structural and functional characteristics that define effective therapeutic uses of IFN-β, and further disclose a diverse, additional array of IFN-β agents and functional variants and analogs of IFN-β (including, but not limited to, natural or recombinant mutant forms of IFN-β, chemically or biosynthetically modified derivatives or variants of IFN-β and polypeptide and small molecule drug mimetics of IFN-β) that are also useful within the invention.

IFN-β is produced mainly by fibroblasts and some epithelial cell types. The synthesis of IFN-β can be induced by common inducers of interferons including viruses, double-stranded RNA, and micro-organisms. It is also induced by some cytokines such as tumor necrosis factor (TNF) and IL1. In contrast to IFN-α, IFN-β is strictly species-specific. IFN-β derived from other species is inactive in human cells

Within the mucosal delivery formulations and methods of the invention, continuous administration of interferon β to patients with multiple sclerosis permits the use of a lower dose, with subsequent lowering of significant drug related side effects. Because continuous infusion outside the hospital setting is impractical, the mucosal formulations for delivery of IFN-β of the present invention allow one to approximate a continuous administration, with the accrued benefits, including improved patient-to-patient dose variability.

Treatment and Prevention of Hepatitis B: As noted above, the instant invention provides improved and useful methods and compositions for mucosal delivery of IFN-β to prevent and treat hepatitis B infection in mammalian subjects. IFN-β alone or in combination with IFN-α is useful in the treatment of chronic active hepatitis B.

Treatment and Prevention of Childhood Viral Encephalitis: As noted above, the instant invention provides improved and useful methods and compositions for mucosal delivery of IFN-β to prevent and treat severe childhood viral encephalitis in mammalian subjects. A combination treatment of interferon β with acyclovir is more effective than treatment with acyclovir alone.

Treatment and Prevention of Condylomata Acuminata: As noted above, the instant invention provides improved and useful methods and compositions for mucosal delivery of IFN-β to prevent and treat papilloma virus infection in mammalian subjects. IFN-β is used for treatment of condyloma acuminata (genital or venereal warts caused by papilloma virus infection), papillomavirus warts of the larynx and skin (common warts). It is also suitable for the prophylactic use following surgical removal of large condylomas.

Treatment and Prevention of Malignant Tumors: Within the mucosal delivery formulations and methods of the invention, IFN-β is a lipophilic molecule that is particularly useful for local tumor therapy due to its specific pharmacokinetics. Head and neck squamous carcinomas, mammary and cervical carcinomas, and also malignant melanomas respond well to treatment with IFN-β. IFN-β is useful for the adjuvant therapy of malignant melanomas with a high potential for metastasis. Response rates are improved by combining IFN-β with antineoplastic agents or other cytokines.

Treatment and Prevention of Malignant Glioma: Within the mucosal delivery formulations and methods of the invention, combination therapy with IFN-β, MCNU (Ranimustine), and radiotherapy had a pronounced effect on untreated malignant glioma, with moderate side effects and no substantial effect on patients' general condition. (Wakabayashi, et al., J. Neurooncol, 49: 57-62, 2000)

Methods and Compositions of Delivery: Improved methods and compositions for mucosal administration of interferon-β to mammalian subjects optimize interferon-β dosing schedules. The present invention provides mucosal delivery of interferon-β formulated with one or more mucosal delivery-enhancing agents wherein interferon-β dosage release is substantially normalized and/or sustained for an effective delivery period of interferon-β release ranges from approximately 0.1 to 2.0 hours; 0.4 to 1.5 hours; 0.7 to 1.5 hours; or 1.0 to 1.3 hours; following mucosal administration. The sustained release of interferon-β is achieved may be facilitated by repeated administration of exogenous interferon-β utilizing methods and compositions of the present invention.

Compositions and Methods of Sustained Release: Improved compositions and methods for mucosal administration of interferon-β to mammalian subjects optimize interferon-β dosing schedules. The present invention provides improved mucosal (e.g., nasal) delivery of a formulation comprising interferon-β in combination with one or more mucosal delivery-enhancing agents and an optional sustained release-enhancing agent or agents. Mucosal delivery-enhancing agents of the present invention yield an effective increase in delivery, e.g., an increase in the maximal plasma concentration (Cₘₐₓ) to enhance the therapeutic activity of mucosally-administered interferon-β. A second factor affecting therapeutic activity of interferon-β in the blood plasma and CNS is residence time (RT). Sustained release-enhancing agents, in combination with intranasal delivery-enhancing agents, increase Cₘₐₓ and increase residence time (RT) interferon-β. Polymeric delivery vehicles and other agents and methods of the present invention that yield sustained release-enhancing formulations, for example, polyethylene glycol (PEG), are disclosed herein. The present invention provides an improved interferon-β delivery method and dosage form for treatment of symptoms related to interferon-β deficiency in mammalian subjects.

Maintenance of Basal Levels of Interferon-β: Improved compositions and methods for mucosal administration of interferon-β to mammalian subjects optimize interferon-β dosing schedules. The present invention provides improved nasal mucosal delivery of a formulation comprising interferon-β and intranasal delivery-enhancing agents in combination with subcutaneous and intramuscular administration of interferon-β. Formulations and methods of the present invention maintain relatively consistent basal levels of interferon-β, for example throughout a 2 to 24 hour, 4-16 hour, or 8-12 hour period following a single dose administration or attended by a multiple dosing regimen of 2-6 sequential administrations, often such that biological markers including neopterin and beta-2 microglobulin or 2,5-oligoadenylate synthetase are maintained at therapeutic levels at all times. Maintenance of basal levels of interferon-β is particularly useful for treatment and prevention of disease, for example, multiple sclerosis, without unacceptable adverse side effects.

Interferon β is produced by various cell types including fibroblasts and macrophages. Interferon β exerts its biological effects by binding to specific receptors on the surface of human cells. This binding initiates a complex cascade of intracellular events that leads to the expression of gene products and markers, for example, 2′,5′ oligoadenylate synthetase (2′,5′-OAS), neopterin, and β₂-microglobulin. These markers have been used to monitor the biological activity of interferon β-1a in humans. Induction of the biological response markers roughly correlates with serum activity levels of interferon β. These biological markers roughly peak 48 hours after administering a intramuscular or subcutaneous dose of interferon β and remain elevated for 4 days. After a intramuscular dose serum levels of interferon β peak about 3 to 15 hours after dosing. The elimination half-life is around 10 hours.

The effectiveness of interferon β is related to the increases in these biological markers. The doses chosen for clinical trials of Avonex® were based on the level of increase in β₂-microglobulin. 6 MIU (30 μg). The recommended dose of Avonex® is 30 μg injected intramuscularly once a week.

For example, interferon β at a 30 μg dose given intramuscularly once weekly would typically be an effective initial dose. The improved nasal mucosal delivery of a formulation comprising interferon-β and intranasal delivery-enhancing agents of the present invention at a dose of 60 to 120 μg per day would typically be given to sustain the biological markers beyond 4 days.

Within the mucosal delivery formulations and methods of the invention, the interferon-β is frequently combined or coordinately administered with a suitable carrier or vehicle for mucosal delivery. As used herein, the term “carrier” means a pharmaceutically acceptable solid or liquid filler, diluent or encapsulating material. A water-containing liquid carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories, can be found in the U.S. Pharmacopeia National Formulary, pp. 1857-1859, 1990. Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other non toxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator. Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like. The amount of active ingredient that can be combined with the carrier materials to produce a single dosage form will vary depending upon the particular mode of administration.

The mucosal formulations of the invention are generally sterile, particulate free and stable for pharmaceutical use. As used herein, the term “particulate free” means a formulation that meets the requirements of the USP specification for small volume parenteral solutions. The term “stable” means a formulation that fulfills all chemical and physical specifications with respect to identity, strength, quality, and purity that have been established according to the principles of Good Manufacturing Practice, as set forth by appropriate governmental regulatory bodies.

Within the mucosal delivery compositions and methods of the invention, various delivery-enhancing agents are employed which enhance delivery of interferon-β into or across a mucosal surface. In this regard, delivery of interferon-β across the mucosal epithelium can occur “transcellularly” or “paracellularly”. The extent to which these pathways contribute to the overall flux and bioavailability of the interferon-β depends upon the environment of the mucosa, the physico-chemical properties the active agent, and on the properties of the mucosal epithelium. Paracellular transport involves only passive diffusion, whereas transcellular transport can occur by passive, facilitated or active processes. Generally, hydrophilic, passively transported, polar solutes diffuse through the paracellular route, while more lipophilic solutes use the transcellular route. Absorption and bioavailability (e.g., as reflected by a permeability coefficient or physiological assay), for diverse, passively and actively absorbed solutes, can be readily evaluated, in terms of both paracellular and transcellular delivery components, for any selected interferon-β within the invention. These values can be determined and distinguished according to well known methods, such as in vitro epithelial cell culture permeability assays (see, e.g., Hilgers, et al., Pharm. Res. 7:902-910, 1990; Wilson et al., J. Controlled Release 11:25-40, 1990; Artursson. I., Pharm. Sci. 79:476-82, 1990; Cogburn et al., Pharm. Res. 8:210-216, 1991; Pade et al., Pharmaceutical Research 14:1210-1215, 1997).

For passively absorbed drugs, the relative contribution of paracellular and transcellular pathways to drug transport depends upon the pKa, partition coefficient, molecular radius and charge of the drug, the pH of the luminal environment in which the drug is delivered, and the area of the absorbing surface. The paracellular route represents a relatively small fraction of accessible surface area of the nasal mucosal epithelium. In general terms, it has been reported that cell membranes occupy a mucosal surface area that is a thousand times greater than the area occupied by the paracellular spaces. Thus, the smaller accessible area, and the size- and charge-based discrimination against macromolecular permeation would suggest that the paracellular route could be a generally less favorable route than transcellular delivery for drug transport. Surprisingly, the methods and compositions of the invention provide for significantly enhanced transport of biotherapeutics into and across mucosal epithelia via the paracellular route. Therefore, the methods and compositions of the invention successfully target both paracellular and transcellular routes, alternatively or within a single method or composition.

As used herein, “mucosal delivery-enhancing agents” include agents which enhance the release or solubility (e.g., from a formulation delivery vehicle), diffusion rate, penetration capacity and timing, uptake, residence time, stability, effective half-life, peak or sustained concentration levels, clearance and other desired mucosal delivery characteristics (e.g., as measured at the site of delivery, or at a selected target site of activity such as the bloodstream or central nervous system) of interferon-β or other biologically active compound(s). Enhancement of mucosal delivery can thus occur by any of a variety of mechanisms, for example by increasing the diffusion, transport, persistence or stability of interferon-β, increasing membrane fluidity, modulating the availability or action of calcium and other ions that regulate intracellular or paracellular permeation, solubilizing mucosal membrane components (e.g., lipids), changing non-protein and protein sulfhydryl levels in mucosal tissues, increasing water flux across the mucosal surface, modulating epithelial junctional physiology, reducing the viscosity of mucus overlying the mucosal epithelium, reducing mucociliary clearance rates, and other mechanisms.

As used herein, a “mucosally effective amount of interferon-β” contemplates effective mucosal delivery of interferon-β to a target site for drug activity in the subject that may involve a variety of delivery or transfer routes. For example, a given active agent may find its way through clearances between cells of the mucosa and reach an adjacent vascular wall, while by another route the agent may, either passively or actively, be taken up into mucosal cells to act within the cells or be discharged or transported out of the cells to reach a secondary target site, such as the systemic circulation. The methods and compositions of the invention may promote the translocation of active agents along one or more such alternate routes, or may act directly on the mucosal tissue or proximal vascular tissue to promote absorption or penetration of the active agent(s). The promotion of absorption or penetration in this context is not limited to these mechanisms.

As used herein “peak concentration (Cₘₐₓ) of interferon-β in a blood plasma”, “area under concentration vs. time curve (AUC) of interferon-β in a blood plasma”, “time to maximal plasma concentration (tₘₐₓ) of interferon-β in a blood plasma” are pharmacokinetic parameters known to one skilled in the art. (Laursen et al., Eur. J. Endocrinology, 135: 309-315, 1996.) The “concentration vs. time curve” measures the concentration of interferon-β in a blood serum of a subject vs. time after administration of a dosage of interferon-β to the subject either by intranasal, subcutaneous, or other parenteral route of administration. “Cₘₐₓ” is the maximum concentration of interferon-β in the blood serum of a subject following a single dosage of interferon-β to the subject. “tₘₐₓ” is the time to reach maximum concentration of interferon-β in a blood serum of a subject following administration of a single dosage of interferon-β to the subject.

As used herein, “area under concentration vs. time curve (AUC) of interferon-β in a blood plasma” is calculated according to the linear trapezoidal rule and with addition of the residual areas. A decrease of 23% or an increase of 30% between two dosages would be detected with a probability of 90% (type II error β=10%). The “delivery rate” or “rate of absorption” is estimated by comparison of the time (tₘₐₓ) to reach the maximum concentration (Cₘₐₓ). Both Cₘₐₓ and tₘₐₓ are analyzed using non-parametric methods. Comparisons of the pharmacokinetics of subcutaneous, intravenous and intranasal interferon-β administrations were performed by analysis of variance (ANOVA). For pairwise comparisons a Bonferroni-Holmes sequential procedure was used to evaluate significance. The dose-response relationship between the three nasal doses was estimated by regression analysis. P<0.05 was considered significant. Results are given as mean values +/−SEM. (Laursen et al., 1996.)

As used herein, “pharmacokinetic markers” include any accepted biological marker that is detectable in an in vitro or in vivo system useful for modeling pharmacokinetics of mucosal delivery of one or more interferon-β compounds, or other interferon beta(s) disclosed herein, wherein levels of the marker(s) detected at a desired target site following administration of the interferon-β compound(s) according to the methods and formulations herein, provide a reasonably correlative estimate of the level(s) of the interferon-β compound(s) delivered to the target site. Among many art-accepted markers in this context are substances induced at the target site by adminstration of the interferon-β compound(s) or orther interferon beta(s). For example, nasal mucosal delivery of an effective amount of one or more interferon-β compounds according to the invention stimulates an immunologic response in the subject measurable by production of pharmacokinetic markers that include, but are not limited to, neopterin and β₂-microglobulin.

While the mechanism of absorption promotion may vary with different intranasal delivery-enhancing agents of the invention, useful reagents in this context will not substantially adversely affect the mucosal tissue and will be selected according to the physicochemical characteristics of the particular interferon-β or other active or delivery-enhancing agent. In this context, delivery-enhancing agents that increase penetration or permeability of mucosal tissues will often result in some alteration of the protective permeability barrier of the mucosa. For such delivery-enhancing agents to be of value within the invention, it is generally desired that any significant changes in permeability of the mucosa be reversible within a time frame appropriate to the desired duration of drug delivery. Furthermore, there should be no substantial, cumulative toxicity, nor any permanent deleterious changes induced in the barrier properties of the mucosa with long-term use.

Within certain aspects of the invention, absorption-promoting agents for coordinate administration or combinatorial formulation with interferon-β of the invention are selected from small hydrophilic molecules, including but not limited to, dimethyl sulfoxide (DMSO), dimethylformamide, ethanol, propylene glycol, and the 2-pyrrolidones. Alternatively, long-chain amphipathic molecules, for example, deacylmethyl sulfoxide, azone, sodium lauryl sulfate, oleic acid, and the bile salts, may be employed to enhance mucosal penetration of the interferon-β. In additional aspects, surfactants (e.g., polysorbates) are employed as adjunct compounds, processing agents, or formulation additives to enhance intranasal delivery of the interferon-β. These penetration-enhancing agents typically interact at either the polar head groups or the hydrophilic tail regions of molecules that comprise the lipid bilayer of epithelial cells lining the nasal mucosa (Barry, Pharmacology of the Skin, Vol. 1, pp. 121-137, Shroot et al., Eds., Karger, Basel, 1987; and Barry, J. controlled Release 6:85-97, 1987). Interaction at these sites may have the effect of disrupting the packing of the lipid molecules, increasing the fluidity of the bilayer, and facilitating transport of the interferon-β across the mucosal barrier. Interaction of these penetration enhancers with the polar head groups may also cause or permit the hydrophilic regions of adjacent bilayers to take up more water and move apart, thus opening the paracellular pathway to transport of the interferon-β. In addition to these effects, certain enhancers may have direct effects on the bulk properties of the aqueous regions of the nasal mucosa. Agents such as DMSO, polyethylene glycol, and ethanol can, if present in sufficiently high concentrations in delivery environment (e.g., by pre-administration or incorporation in a therapeutic formulation), enter the aqueous phase of the mucosa and alter its solubilizing properties, thereby enhancing the partitioning of the interferon-β from the vehicle into the mucosa.

Additional mucosal delivery-enhancing agents that are useful within the coordinate administration and processing methods and combinatorial formulations of the invention include, but are not limited to, mixed micelles; enamines; nitric oxide donors (e.g., S-nitroso-N-acetyl-DL-penicillamine, NOR1, NOR4—which are preferably co-administered with an NO scavenger such as carboxy-PITO or doclofenac sodium); sodium salicylate; glycerol esters of acetoacetic acid (e.g., glyceryl-1,3-diacetoacetate or 1,2-isopropylideneglycerine-3-acetoacetate); and other release-diffusion or intra- or trans-epithelial penetration-promoting agents that are physiologically compatible for mucosal delivery. Other absorption-promoting agents are selected from a variety of carriers, bases and excipients that enhance mucosal delivery, stability, activity or trans-epithelial penetration of the interferon-β. These include, inter alia, clyclodextrins and β-cyclodextrin derivatives (e.g., 2-hydroxypropyl-β-cyclodextrin and heptakis(2,6-di-O-methyl-β-cyclodextrin). These compounds, optionally conjugated with one or more of the active ingredients and further optionally formulated in an oleaginous base, enhance bioavailability in the mucosal formulations of the invention. Yet additional absorption-enhancing agents adapted for mucosal delivery include medium-chain fatty acids, including mono- and diglycerides (e.g., sodium caprate—extracts of coconut oil, Capmul), and triglycerides (e.g., amylodextrin, Estaram 299, Miglyol 810).

The mucosal therapeutic and prophylactic compositions of the present invention may be supplemented with any suitable penetration-promoting agent that facilitates absorption, diffusion, or penetration of interferon-β across mucosal barriers. The penetration promoter may be any promoter that is pharmaceutically acceptable. Thus, in more detailed aspects of the invention compositions are provided that incorporate one or more penetration-promoting agents selected from sodium salicylate and salicylic acid derivatives (acetyl salicylate, choline salicylate, salicylamide, etc.); amino acids and salts thereof (e.g. monoaminocarboxlic acids such as glycine, alanine, phenylalanine, proline, hydroxyproline, etc.; hydroxyamino acids such as serine; acidic amino acids such as aspartic acid, glutamic acid, etc; and basic amino acids such as lysine etc—inclusive of their alkali metal or alkaline earth metal salts); and N-acetylamino acids (N-acetylalanine, N-acetylphenylalanine, N-acetylserine, N-acetylglycine, N-acetyllysine, N-acetylglutamic acid, N-acetylproline, N-acetylhydroxyproline, etc.) and their salts (alkali metal salts and alkaline earth metal salts). Also provided as penetration-promoting agents within the methods and compositions of the invention are substances which are generally used as emulsifiers (e.g. sodium oleyl phosphate, sodium lauryl phosphate, sodium lauryl sulfate, sodium myristyl sulfate, polyoxyethylene alkyl ethers, polyoxyethylene alkyl esters, etc.), caproic acid, lactic acid, malic acid and citric acid and alkali metal salts thereof, pyrrolidonecarboxylic acids, alkylpyrrolidonecarboxylic acid esters, N-alkylpyrrolidones, proline acyl esters, and the like.

Within various aspects of the invention, improved nasal mucosal delivery formulations and methods are provided that allow delivery of interferon-β and other therapeutic agents within the invention across mucosal barriers between administration and selected target sites. Certain formulations are specifically adapted for a selected target cell, tissue or organ, or even a particular disease state. In other aspects, formulations and methods provide for efficient, selective endo- or transcytosis of interferon-β specifically routed along a defined intracellular or intercellular pathway. Typically, the interferon-β is efficiently loaded at effective concentration levels in a carrier or other delivery vehicle, and is delivered and maintained in a stabilized form, e.g., at the nasal mucosa and/or during passage through intracellular compartments and membranes to a remote target site for drug action (e.g., the blood stream or a defined tissue, organ, or extracellular compartment). The interferon-β may be provided in a delivery vehicle or otherwise modified (e.g., in the form of a prodrug), wherein release or activation of the interferon-β is triggered by a physiological stimulus (e.g. pH change, lysosomal enzymes, etc.) Often, the interferon-β is pharmacologically inactive until it reaches its target site for activity. In most cases, the interferon-β and other formulation components are non-toxic and non-immunogenic. In this context, carriers and other formulation components are generally selected for their ability to be rapidly degraded and excreted under physiological conditions. At the same time, formulations are chemically and physically stable in dosage form for effective storage.

A variety of additives, diluents, bases and delivery vehicles are provided within the invention that effectively control water content to enhance protein stability. These reagents and carrier materials effective as anti-aggregation agents in this sense include, for example, polymers of various functionalities, such as polyethylene glycol, dextran, diethylaminoethyl dextran, and carboxymethyl cellulose, which significantly increase the stability and reduce the solid-phase aggregation of peptides and proteins admixed therewith or linked thereto. In some instances, the activity or physical stability of proteins can also be enhanced by various additives to aqueous solutions of the peptide or protein drugs. For example, additives, such as polyols (including sugars), amino acids, and various salts may be used.

Certain additives, in particular sugars and other polyols, also impart significant physical stability to dry, e.g., lyophilized proteins. These additives can also be used within the invention to protect the proteins against aggregation not only during lyophilization but also during storage in the dry state. For example sucrose and Ficoll 70 (a polymer with sucrose units) exhibit significant protection against peptide or protein aggregation during solid-phase incubation under various conditions. These additives may also enhance the stability of solid proteins embedded within polymer matrices.

Yet additional additives, for example sucrose, stabilize proteins against solid-state aggregation in humid atmospheres at elevated temperatures, as may occur in certain sustained-release formulations of the invention. These additives can be incorporated into polymeric melt processes and compositions within the invention. For example, polypeptide microparticles can be prepared by simply lyophilizing or spray drying a solution containing various stabilizing additives described above. Sustained release of unaggregated peptides and proteins can thereby be obtained over an extended period of time.

Various additional preparative components and methods, as well as specific formulation additives, are provided herein which yield formulations for mucosal delivery of aggregation-prone peptides and proteins, wherein the peptide or protein is stabilized in a substantially pure, unaggregated form. A range of components and additives are contemplated for use within these methods and formulations. Exemplary of these anti-aggregation agents are linked dimers of cyclodextrins (CDs), which selectively bind hydrophobic side chains of polypeptides (see, e.g., Breslow, et al., J. Am. Chem. Soc. 120:3536-3537; Maletic, et al., Angew. Chem. Int. Ed. Engl. 35:1490-1492. These CD dimers have been found to bind to hydrophobic patches of proteins in a manner that significantly inhibits aggregation (Leung et al., Proc. Nat.l Acad. Sci. USA 97:5050-5053, 2000). This inhibition is selective with respect to both the CD dimer and the protein involved. Such selective inhibition of protein aggregation provides additional advantages within the intranasal delivery methods and compositions of the invention. Additional agents for use in this context include CD trimers and tetramers with varying geometries controlled by the linkers that specifically block aggregation of peptides and proteins (Breslow et al., J. Am. Chem. Soc. 118:11678-11681, 1996; Breslow et al., PNAS USA 94:11156-11158, 1997; Breslow et al., Tetrahedron Lett. 2887-2890, 1998).

Yet additional anti-aggregation agents and methods for incorporation within the invention involve the use of peptides and peptide mimetics to selectively block protein-protein interactions. In one aspect, the specific binding of hydrophobic side chains reported for CD multimers is extended to proteins via the use of peptides and peptide mimetics that similarly block protein aggregation. A wide range of suitable methods and anti-aggregation agents are available for incorporation within the compositions and procedures of the invention (Zutshi et al., Curr. Opin. Chem. Biol. 2:62-66, 1998; Daugherty et al., J. Am. Chem. Soc. 121:4325-4333, 1999: Zutshi et al., J. Am. Chem. Soc. 119:4841-4845, 1997; Ghosh et al, Chem. Biol. 5:439-445, 1997; Hamuro et al., Angew. Chem. Int. Ed. Engl. 36:2680-2683, 1997; Alberg et al., Science 262:248-250, 1993; Tauton et al., J. Am. Chem. Soc. 118:10412-10422, 1996; Park et al., J. Am. Chem. Soc. 121:8-13, 1999; Prasanna et al., Biochemistry 37:6883-6893, 1998; Tiley et al., J. Am. Chem. Soc. 119:7589-7590, 1997; Judice et al., PNAS, USA 94:13426-13430, 1997; Fan et al., J. Am. Chem. Soc. 120:8893-8894, 1998; Gamboni et al., Biochemistry 37:12189-12194, 1998). Other techniques in peptide and protein engineering disclosed herein will further reduce the extent of protein aggregation and instability in mucosal delivery methods and formulations of the invention. One example of a useful method for peptide or protein modification in this context is PEGylation. The stability and aggregation problems of polypeptide drugs can be significantly improved by covalently conjugating water-soluble polymers such as PEG with the polypeptide.

Enzyme inhibitors for use within the invention are selected from a wide range of non-protein inhibitors that vary in their degree of potency and toxicity (see, e.g., L. Stryer, Biochemistry, WH Freeman and Company, New York, N.Y., 1988). As described in further detail below, immobilization of these adjunct agents to matrices or other delivery vehicles, or development of chemically modified analogues, may be readily implemented to reduce or even eliminate toxic effects, when they are encountered. Among this broad group of candidate enzyme inhibitors for use within the invention are organophosphorous inhibitors, such as diisopropylfluorophosphate (DFP) and phenylmethylsulfonyl fluoride (PMSF), which are potent, irreversible inhibitors of serine proteases (e.g., trypsin and chymotrypsin). The additional inhibition of acetylcholinesterase by these compounds makes them highly toxic in uncontrolled delivery settings (L. Stryer, Biochemistry, WH Freeman and Company, New York, N.Y., 1988). Another candidate inhibitor, 4-(2-Aminoethyl)-benzenesulfonyl fluoride (AEBSF), has an inhibitory activity comparable to DFP and PMSF, but it is markedly less toxic. (4-Aminophenyl)-methanesulfonyl fluoride hydrochloride (APMSF) is another potent inhibitor of trypsin, but is toxic in uncontrolled settings. In contrast to these inhibitors, 4-(4-isopropylpiperadinocarbonyl)phenyl 1,2,3,4,-tetrahydro-1-naphthoate methanesulphonate (FK-448) is a low toxic substance, representing a potent and specific inhibitor of chymotrypsin. Further representatives of this non-protein group of inhibitor candidates, and also exhibiting low toxic risk, are camostat mesilate (N,N′-dimethyl carbamoylmethyl-p-(p′-guanidino-benzoyloxy)phenylacetate methane-sulphonate).

Yet another type of enzyme inhibitory agent for use within the methods and compositions of the invention are amino acids and modified amino acids that interfere with enzymatic degradation of specific therapeutic compounds. For use in this context, amino acids and modified amino acids are substantially non-toxic and can be produced at a low cost. However, due to their low molecular size and good solubility, they are readily diluted and absorbed in mucosal environments. Nevertheless, under proper conditions, amino acids can act as reversible, competitive inhibitors of protease enzymes. Certain modified amino acids can display a much stronger inhibitory activity. A desired modified amino acid in this context is known as a ‘transition-state’ inhibitor. The strong inhibitory activity of these compounds is based on their structural similarity to a substrate in its transition-state geometry, while they are generally selected to have a much higher affinity for the active site of an enzyme than the substrate itself. Transition-state inhibitors are reversible, competitive inhibitors. Examples of this type of inhibitor are α-aminoboronic acid derivatives, such as boro-leucine, boro-valine and boro-alanine. The boron atom in these derivatives can form a tetrahedral boronate ion that is believed to resemble the transition state of peptides during their hydrolysis by aminopeptidases. These amino acid derivatives are potent and reversible inhibitors of aminopeptidases and it is reported that boro-leucine is more than 100-times more effective in enzyme inhibition than bestatin and more than 1000-times more effective than puromycin. Another modified amino acid for which a strong protease inhibitory activity has been reported is N-acetylcysteine, which inhibits enzymatic activity of aminopeptidase N. This adjunct agent also displays mucolytic properties that can be employed within the methods and compositions of the invention to reduce the effects of the mucus diffusion barrier.

Agents suitable for protease inhibition are the complexing agents EDTA and DTPA as coordinately administered or combinatorially formulated adjunct agents, in suitable concentration, will be sufficient to inhibit selected proteases to thereby enhance intranasal delivery of interferon betas according to the invention. Further representatives of-this class of inhibitory agents are EGTA, 1,10-phenanthroline and hydroxychinoline. In addition, due to their propensity to chelate divalent cations, these and other complexing agents are useful within the invention as direct, absorption-promoting agents. As noted in more detail elsewhere herein, it is also contemplated to use various polymers, particularly mucoadhesive polymers, as enzyme inhibiting agents within the coordinate administration, multi-processing and/or combinatorial formulation methods and compositions of the invention. For example, poly(acrylate) derivatives, such as poly(acrylic acid) and polycarbophil, can affect the activity of various proteases, including trypsin, chymotrypsin. The inhibitory effect of these polymers may also be based on the complexation of divalent cations such as Ca² and Zn². It is further contemplated that these polymers may serve as conjugate partners or carriers for additional enzyme inhibitory agents, as described above. For example, a chitosan-EDTA conjugate has been developed and is useful within the invention that exhibits a strong inhibitory effect towards the enzymatic activity of zinc-dependent proteases. The mucoadhesive properties of polymers following covalent attachment of other enzyme inhibitors in this context are not expected to be substantially compromised, nor is the general utility of such polymers as a delivery vehicle for interferon betas within the invention expected to be diminished. On the contrary, the reduced distance between the delivery vehicle and mucosal surface afforded by the mucoadhesive mechanism will minimize presystemic metabolism of the active agent, while the covalently bound enzyme inhibitors remain concentrated at the site of drug delivery, minimizing undesired dilution effects of inhibitors as well as toxic and other side effects caused thereby. In this manner, the effective amount of a coordinately administered enzyme inhibitor can be reduced due to the exclusion of dilution effects.

Ciliostatic Agents and Methods

Because the self-cleaning capacity of certain mucosal tissues (e.g., nasal mucosal tissues) by mucociliary clearance is necessary as a protective function (e.g., to remove dust, allergens, and bacteria), it has been generally considered that this function should not be substantially impaired by mucosal medications. Mucociliary transport in the respiratory tract is a particularly important defense mechanism against infections. To achieve this function, ciliary beating in the nasal and airway passages moves a layer of mucus along the mucosa to removing inhaled particles and microorganisms.

Various reports show that mucociliary clearance can be impaired by mucosally administered drugs, as well as by a wide range of formulation additives including penetration enhancers and preservatives. For example, ethanol at concentrations greater than 2% has been shown to reduce the in vitro ciliary beating frequency. This may be mediated in part by an increase in membrane permeability that indirectly enhances flux of calcium ion which, at high concentration, is ciliostatic, or by a direct effect on the ciliary axoneme or actuation of regulatory proteins involved in a ciliary arrest response. Exemplary preservatives (methyl-p-hydroxybenzoate (0.02% and 0.15%), propyl-p-hydroxybenzoate (0.02%), and chlorobutanol (0.5%)) reversibly inhibit ciliary activity in a frog palate model. Other common additives (EDTA (0.1%), benzalkoniuin chloride (0.01%), chlorhexidine (0.01%), phenylinercuric nitrate (0.002%), and phenylmercuric borate (0.002%), have been reported to inhibit mucociliary transport irreversibly. In addition, several penetration enhancers including STDHF, laureth-9, deoxycholate, deoxycholic acid, taurocholic acid, and glycocholic acid have been reported to inhibit ciliary activity in model systems.

Despite the potential for adverse effects on mucociliary clearance attributed to ciliostatic factors, ciliostatic agents nonetheless find use within the methods and compositions of the invention to increase the residence time of mucosally (e.g., intranasally) administered interferon-β peptides, proteins, analogs and mimetics, and other interferon betas disclosed herein. In particular, the delivery these agents within the methods and compositions of the invention is significantly enhanced in certain aspects by the coordinate administration or combinatorial formulation of one or more ciliostatic agents that function to reversibly inhibit ciliary activity of mucosal cells, to provide for a temporary, reversible increase in the residence time of the mucosally administered active agent(s). For use within these aspects of the invention, the foregoing ciliostatic factors, either specific or indirect in their activity, are all candidates for successful employment as ciliostatic agents in appropriate amounts (depending on concentration, duration and mode of delivery) such that they yield a transient (i.e., reversible) reduction or cessation of mucociliary clearance at a mucosal site of administration to enhance delivery of interferon-β peptides, proteins, analogs and mimetics, and other interferon betas disclosed herein, without unacceptable adverse side effects.

Surface Active Agents and Methods

Within more detailed aspects of the invention, one or more membrane penetration-enhancing agents may be employed within a mucosal delivery method or formulation of the invention to enhance mucosal delivery of interferon-β peptides, proteins, analogs and mimetics, and other interferon betas disclosed herein. Membrane penetration enhancing agents in this context can be selected from: (i) a surfactant, (ii) a bile salt, (ii) a phospholipid additive, mixed micelle, liposome, or carrier, (iii) an alcohol, (iv) an enamine, (v) an NO donor compound, (vi) a long-chain amphipathic molecule (vii) a small hydrophobic penetration enhancer; (viii) sodium or a salicylic acid derivative; (ix) a glycerol ester of acetoacetic acid (x) a clyclodextrin or beta-cyclodextrin derivative, (xi) a medium-chain fatty acid, (xii) a chelating agent, (xiii) an amino acid or salt thereof, (xiv) an N-acetylamino acid or salt thereof, (xv) an inhibitor of fatty acid synthesis, or (xvi) an inhibitor of cholesterol synthesis; or (xi) any combination of the membrane penetration enhancing agents recited in (i)-(xvi)

Certain surface-active agents are readily incorporated within the mucosal delivery formulations and methods of the invention as mucosal absorption enhancing agents. These agents, which may be coordinately administered or combinatorially formulated with interferon-β peptides, proteins, analogs and mimetics, and other interferon betas disclosed herein, may be selected from a broad assemblage of known surfactants. Surfactants, which generally fall into three classes: (1) nonionic polyoxyethylene ethers; (2) bile salts such as sodium glycocholate (SGC) and deoxycholate (DOC); and (3) derivatives of fusidic acid such as sodium taurodihydrofusidate (STDHF).

In certain embodiments of the invention, interferon beta and a permeabilizing agent as described above are administered in combination with one or more mucosal delivery-enhancing agent(s). In more detailed embodiments of the inventions, the pharmaceutical compositions noted above are formulated for intranasal administration. In exemplary embodiments, the formulations are provided as an intranasal spray or powder. To enhance intranasal administration, these formulations may combine the interferon beta and permeabilizing agent with one or more intranasal delivery-enhancing agents selected from:
(a) an aggregation inhibitory agent;
(b) a charge modifying agent;
(c) a pH control agent;
(d) a degradative enzyme inhibitory agent;
(e) a mucolytic or mucus clearing agent;
(f) a ciliostatic agent;
(g) a membrane penetration-enhancing agent selected from (i) a surfactant, (ii) a bile salt, (ii) a phospholipid additive, mixed micelle, liposome, or carrier, (iii) an alcohol, (iv) an enamine, (v) an NO donor compound, (vi) a long-chain amphipathic molecule (vii) a small hydrophobic penetration enhancer; (viii) sodium or a salicylic acid derivative; (ix) a glycerol ester of acetoacetic acid (x) a cyclodextrin or beta-cyclodextrin derivative, (xi) a medium-chain fatty acid, (xii) a chelating agent, (xiii) an amino acid or salt thereof, (xiv) an N-acetylamino acid or salt thereof, (xv) an inhibitor of fatty acid synthesis, or (xvi) an inhibitor of cholesterol synthesis; or (xvii) any combination of the membrane penetration enhancing agents recited in (i)-(xvii);
(h) a second modulatory agent of epithelial junction physiology;
(i) a vasodilator agent;
(j) a selective transport-enhancing agent; and
(k) a stabilizing delivery vehicle, carrier, support or complex-forming species with which the interferon beta is effectively combined, associated, contained, encapsulated or bound resulting in stabilization of the active agent for enhanced intranasal delivery, wherein said one or more intranasal delivery-enhancing agents comprises any one or combination of two or more of said intranasal delivery-enhancing agents recited in (a)-(k), and wherein the formulation of said interferon beta with said one or more intranasal delivery-enhancing agents provides for increased bioavailability of the interferon beta delivered to a nasal mucosal surface of a mammalian subject.

In alternate embodiments of the invention, the pharmaceutical compositions comprising a permeabilizing agent and a interferon beta, wherein said composition is free of a stabilizer that is a protein or polypeptide, are effective following mucosal administration to yield enhanced bioavailability by yielding an area under concentration curve (AUC) of the interferon beta in a blood plasma or cerebral spinal fluid (CNS) of the subject that is about 25% or greater compared to an AUC of the interferon beta in blood plasma or CNS following intramuscular injection of an equivalent concentration or dose of the active agent to the subject. In certain embodiments, the pharmaceutical compositions yield an area under concentration curve (AUC) of the interferon beta in a blood plasma or cerebral spinal fluid (CNS) of the subject that is about 50% or greater compared to an AUC of the interferon beta in blood plasma or CNS following intramuscular injection of an equivalent concentration or dose of the active agent to the subject.

In additional embodiments of the invention, the pharmaceutical compositions comprising a permeabilizing agent and a interferon beta are effective following mucosal administration to yield enhanced bioavailability by yielding a time to maximal plasma concentration (tₘₐₓ) of said interferon beta in a blood plasma or cerebral spinal fluid (CNS) of the subject between about 0.1 to 1.0 hours. In certain embodiments, the compositions yield a time to maximal plasma concentration (tₘₐₓ) of the interferon beta in a blood plasma or cerebral spinal fluid (CNS) of the subject between about 0.2 to 0.5 hours.

In other embodiments of the invention, the pharmaceutical compositions comprising a permeabilizing agent and a interferon beta are effective following mucosal administration to yield enhanced bioavailability of the active agent in the CNS, for example by yielding a peak concentration of the interferon beta in a CNS tissue or fluid of the subject that is 10% or greater compared to a peak concentration of the interferon beta in a blood plasma of the subject (e.g., wherein the CNS and plasma concentration is measured contemporaneously in the same subject following the mucosal administration). In certain embodiments, compositions of the invention yield a peak concentration of the interferon beta in a CNS tissue or fluid of the subject that is 20%, 40%, or greater compared to a peak concentration of the active agent in a blood plasma of the subject.

Bioadhesive Delivery Vehicles and Methods

In certain aspects of the invention, the combinatorial formulations and/or coordinate administration methods herein incorporate an effective amount of a nontoxic bioadhesive as an adjunct compound or carrier to enhance mucosal delivery of one or more interferon beta(s).

A particularly useful bioadhesive agent within the coordinate administration, and/or combinatorial formulation methods and compositions of the invention is chitosan, as well as its analogs and derivatives. Chitosan is a non-toxic, biocompatible and biodegradable polymer that is widely used for pharmaceutical and medical applications because of its favorable properties of low toxicity and good biocompatibility (Yomota, Pharm. Tech. Japan 10:557-564, 1994). It is a natural polyaminosaccharide prepared from chitin by N-deacetylation with alkali. Furthermore, chitosan has been reported to promote absorption of small polar molecules and peptide and protein drugs through nasal mucosa in animal models and human volunteers

As used within the methods and compositions of the invention, chitosan increases the retention of interferon-β peptides, proteins, analogs and mimetics, and other interferon betas disclosed herein at a mucosal site of application. This is may be mediated in part by a positive charge characteristic of chitosan, which may influence epithelial permeability even after physical removal of chitosan from the surface. As with other bioadhesive gels provided herein, the use of chitosan can reduce the frequency of application and the amount of interferon beta administered while yielding an effective delivery amount or dose. This mode of administration can also improve patient compliance and acceptance. The occlusion and lubrication of chitosan and other bioadhesive gels is expected to reduce the discomfort of inflammatory, allergic and ulcerative conditions of the nasal mucosa.

As further provided herein, the methods and compositions of the invention will optionally include a novel chitosan derivative or chemically modified form of chitosan. One such novel derivative for use within the invention is denoted as β-[1→4]-2-guanidino-2-deoxy-D-glucose polymer (poly-GuD).

Liposomes and Micellar Delivery Vehicles

The coordinate administration methods and combinatorial formulations of the instant invention optionally incorporate effective lipid or fatty acid based carriers, processing agents, or delivery vehicles, to provide improved formulations for mucosal delivery of interferon-β peptides, proteins, analogs and mimetics, and other interferon betas. For example, a variety of formulations and methods are provided for mucosal delivery which comprise one or more of these active agents, such as a peptide or protein, admixed or encapsulated by, or coordinately administered with, a liposome, mixed micellar carrier, or emulsion, to enhance chemical and physical stability and increase the half life of the interferon betas (e.g., by reducing susceptibility to proteolysis, chemical modification and/or denaturation) upon mucosal delivery.

Additional delivery vehicles for use within the invention include long and medium chain fatty acids, as well as surfactant mixed micelles with fatty acids (see, e.g., Muranishi, Crit. Rev. Ther. Drug Carrier Syst. 7:1-33, 1990). Most naturally occurring lipids in the form of esters have important implications with regard to their own transport across mucosal surfaces. Free fatty acids and their monoglycerides which have polar groups attached have been demonstrated in the form of mixed micelles to act on the intestinal barrier as penetration enhancers. This discovery of barrier modifying function of free fatty acids (carboxylic acids with a chain length varying from 12 to 20 carbon atoms) and their polar derivatives has stimulated extensive research on the application of these agents as mucosal absorption enhancers.

For use within the methods of the invention, long chain fatty acids, especially fusogenic lipids (unsaturated fatty acids and monoglycerides such as oleic acid, linoleic acid, linoleic acid, monoolein, etc.) provide useful carriers to enhance mucosal delivery of interferon-β peptides, proteins, analogs and mimetics, and other interferon betas disclosed herein. Medium chain fatty acids (C6 to C12) and monoglycerides have also been shown to have enhancing activity in intestinal drug absorption and can be adapted for use within the mocosal delivery formulations and methods of the invention. In addition, sodium salts of medium and long chain fatty acids are effective delivery vehicles and absorption-enhancing agents for mucosal delivery of interferon betas within the invention. Thus, fatty acids can be employed in soluble forms of sodium salts or by the addition of non-toxic surfactants, e.g., polyoxyethylated hydrogenated castor oil, sodium taurocholate, etc. Mixed micelles of naturally occurring unsaturated long chain fatty acids (oleic acid or linoleic acid) and their monoglycerides with bile salts have been shown to exhibit absorption-enhancing abilities which are basically harmless to the intestinal mucosa (see, e.g., Muranishi, Pharm. Res. 2:108-118, 1985; and Crit. Rev. Ther. drug carrier Syst. 7:1-33, 1990). Other fatty acid and mixed micellar preparations that are useful within the invention include, but are not limited to, Na caprylate (C8), Na caprate (C10), Na laurate (C12) or Na oleate (C18), optionally combined with bile salts, such as glycocholate and taurocholate.

A satisfactory surface-active agent is selected from the group consisting of L-α-phosphatidylcholine didecanoyl (DDPC), polysorbate 20 (Tween 20), polysorbate 80 (Tween 80), polyethylene glycol (PEG), cetyl alcohol, polyvinylpyrolidone (PVP), polyvinyl alcohol (PVA), lanolin alcohol, sphingomyelin, phosphatidylethanolamine and sorbitan monooleate.

Any solubilizing agent can be used but a preferred one is selected from the group consisting of hydroxypropyl-β-cyclodextran, sulfobutylether-β-cyclodextran, methyl-β-cyclodextrin and chitosan.

Examples of chelating agents that can be used in the present invention include deferiprone, deferoxamine, ditiocarb sodium, penicillamine, pentetate calcium trisodium, pentetic acid, succimer, trientine, and EDTA (which includes edetate calcium disodium, edetate disodium, edetate sodium and edetate trisodium).

Pegylation

Additional methods and compositions provided within the invention involve chemical modification of biologically active peptides and proteins by covalent attachment of polymeric materials, for example dextrans, polyvinyl pyrrolidones, glycopeptides, polyethylene glycol and polyamino acids. The resulting conjugated peptides and proteins retain their biological activities and solubility for mucosal administration. In alternate embodiments, interferon-β peptides, proteins, analogs and mimetics, and other biologically active peptides and proteins, are conjugated to polyalkylene oxide polymers, particularly polyethylene glycols (PEG) (see, e.g., U.S. Pat. No. 4,179,337). Numerous reports in the literature describe the potential advantages of pegylated peptides and proteins, which often exhibit increased resistance to proteolytic degradation, increased plasma half-life, increased solubility and decreased antigenicity and immunogenicity (Nucci, et al., Advanced Drug Deliver Reviews 6:133-155, 1991; Lu et al., Int. J. Peptide Protein Res. 43:127-138, 1994).

Formulation and Administration

Mucosal delivery formulations of the present invention comprise the interferon beta to be administered (e.g., one or more of the interferon-β peptides, proteins, analogs and mimetics, and other interferon betas disclosed herein), typically combined together with one or more pharmaceutically acceptable carriers and, optionally, other therapeutic ingredients. The carrier(s) must be “pharmaceutically acceptable” in the sense of being compatible with the other ingredients of the formulation and not eliciting an unacceptable deleterious effect in the subject. Such carriers are described herein above or are otherwise well known to those skilled in the art of pharmacology. Desirably, the formulation should not include substances such as enzymes or oxidizing agents with which the interferon beta to be administered is known to be incompatible. The formulations may be prepared by any of the methods well known in the art of pharmacy.

Compositions according to the present invention are often administered in an aqueous solution as a nasal spray and may be dispensed in spray form by a variety of methods known to those skilled in the art. Examples include nasal actuators produced by Ing. Erich Pfeiffer GmbH, Radolfzell, Germany. See U.S. Pat. No. 4,511,069; U.S. Pat. No. 4,778,810; U.S. Pat. No. 5,203,840; U.S. Pat. No. 5,860,567; U.S. Pat. No. 5,893,484; U.S. Pat. No. 6,227,415; and U.S. Pat. No. 6,364,166. Additional aerosol delivery forms may include, e.g., compressed air-, jet-, ultrasonic-, and piezoelectric nebulizers, which deliver the interferon beta dissolved or suspended in a pharmaceutical solvent, e.g., water, ethanol, or a mixture thereof.

Nasal and pulmonary spray solutions of the present invention typically comprise the drug or drug to be delivered, optionally formulated with a surface active agent, such as a nonionic surfactant (e.g., polysorbate-80), and one or more buffers. In some embodiments of the present invention, the nasal spray solution further comprises a propellant. The pH of the nasal spray solution is optionally between about pH 6.8 and 7.2, but when desired the pH is adjusted to optimize delivery of a charged macromolecular species (e.g., a therapeutic protein or peptide) in a substantially unionized state. The pharmaceutical solvents employed can also be a slightly acidic aqueous buffer (pH 4-6). Suitable buffers for use within these compositions are as described above or as otherwise known in the art. Other components may be added to enhance or maintain chemical stability, including preservatives, surfactants, dispersants, or gases. Suitable preservatives include, but are not limited to, phenol, methyl paraben, paraben, m-cresol, thiomersal, benzylalkonimum chloride, and the like. Suitable surfactants include, but are not limited to, oleic acid, sorbitan trioleate, polysorbates, lecithin, phosphotidyl cholines, and various long chain diglycerides and phospholipids. Suitable dispersants include, but are not limited to, ethylenediaminetetraacetic acid, and the like. Suitable gases include, but are not limited to, nitrogen, helium, chlorofluorocarbons (CFCs), hydrofluorocarbons (HFCs), carbon dioxide, air, and the like.

To formulate compositions for mucosal delivery within the present invention, the interferon beta can be combined with various pharmaceutically acceptable additives, as well as a base or carrier for dispersion of the active agent(s). Desired additives include, but are not limited to, pH control agents, such as arginine, sodium hydroxide, glycine, hydrochloric acid, citric acid, etc. In addition, local anesthetics (e.g., benzyl alcohol), isotonizing agents (e.g., sodium chloride, mannitol, sorbitol), adsorption inhibitors (e.g., Tween 80), solubility enhancing agents (e.g., cyclodextrins and derivatives thereof), stabilizers, and reducing agents (e.g., glutathione) can be included. When the composition for mucosal delivery is a liquid, the tonicity of the formulation, as measured with reference to the tonicity of 0.9% (w/v) physiological saline solution taken as unity, is typically adjusted to a value at which no substantial, irreversible tissue damage will be induced in the nasal mucosa at the site of administration. Generally, the tonicity of the solution is adjusted to a value of about ⅓ to 3, more typically ½ to 2, and most often ¾ to 1.7.

The interferon beta may be dispersed in a base or vehicle, which may comprise a hydrophilic compound having a capacity to disperse the active agent and any desired additives. The base may be selected from a wide range of suitable carriers, including but not limited to, copolymers of polycarboxylic acids or salts thereof, carboxylic anhydrides (e.g. maleic anhydride) with other monomers (e.g. methyl (meth)acrylate, acrylic acid, etc.), hydrophilic vinyl polymers such as polyvinyl acetate, polyvinyl alcohol, polyvinylpyrrolidone, cellulose derivatives such as hydroxymethylcellulose, hydroxypropylcellulose, etc., and natural polymers such as chitosan, collagen, sodium alginate, gelatin, hyaluronic acid, and nontoxic metal salts thereof. Often, a biodegradable polymer is selected as a base or carrier, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters, etc. can be employed as carriers. Hydrophilic polymers and other carriers can be used alone or in combination, and enhanced structural integrity can be imparted to the carrier by partial crystallization, ionic bonding, crosslinking and the like. The carrier can be provided in a variety of forms, including, fluid or viscous solutions, gels, pastes, powders, microspheres and films for direct application to the nasal mucosa. The use of a selected carrier in this context may result in promotion of absorption of the interferon beta.

The interferon beta can be combined with the base or carrier according to a variety of methods, and release of the active agent may be by diffusion, disintegration of the carrier, or associated formulation of water channels. In some circumstances, the active agent is dispersed in microcapsules (microspheres) or nanocapsules (nanospheres) prepared from a suitable polymer, e.g., isobutyl 2-cyanoacrylate (see, e.g., Michael et al., J. Pharmacy Pharmacol. 43: 1-5, 1991), and dispersed in a biocompatible dispersing medium applied to the nasal mucosa, which yields sustained delivery and biological activity over a protracted time.

To further enhance mucosal delivery of pharmaceutical agents within the invention, formulations comprising the active agent may also contain a hydrophilic low molecular weight compound as a base or excipient. Such hydrophilic low molecular weight compounds provide a passage medium through which a water-soluble active agent, such as a physiologically active peptide or protein, may diffuse through the base to the body surface where the active agent is absorbed. The hydrophilic low molecular weight compound optionally absorbs moisture from the mucosa or the administration atmosphere and dissolves the water-soluble active peptide. Exemplary hydrophilic low molecular weight compound include polyol compounds, such as oligo-, di- and monosaccarides such as sucrose, mannitol, lactose, L-arabinose, D-erythrose, D-ribose, D-xylose, D-mannose, D-galactose, lactulose, cellobiose, gentibiose, glycerin and polyethylene glycol. Other examples of hydrophilic low molecular weight compounds useful as carriers within the invention include N-methylpyrrolidone, and alcohols (e.g. oligovinyl alcohol, ethanol, ethylene glycol, propylene glycol, etc.) These hydrophilic low molecular weight compounds can be used alone or in combination with one another or with other active or inactive components of the intranasal formulation.

The compositions of the invention may alternatively contain as pharmaceutically acceptable carriers substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. For solid compositions, conventional nontoxic pharmaceutically acceptable carriers can be used which include, for example, pharmaceutical grades of mannitol, lactose, starch, magnesium stearate, sodium saccharin, talcum, cellulose, glucose, sucrose, magnesium carbonate, and the like.

Therapeutic compositions for administering the interferon beta can also be formulated as a solution, microemulsion, or other ordered structure suitable for high concentration of active ingredients. The carrier can be a solvent or dispersion medium containing, for example, water, ethanol, polyol (for example, glycerol, propylene glycol, and liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants. In many cases, it will be desirable to include isotonic agents, for example, sugars, polyalcohols such as mannitol, sorbitol, or sodium chloride in the composition. Prolonged absorption of the interferon beta can be brought about by including in the composition an agent which delays absorption, for example, monostearate salts and gelatin.

In more detailed aspects of the invention, the interferon beta is stabilized to extend its effective half-life following delivery to the subject, particularly for extending metabolic persistence in an active state within the physiological environment (e.g., at the nasal mucosal surface, in the bloodstream, or within a connective tissue compartment or fluid-filled body cavity

Dosage

For prophylactic and treatment purposes, the interferon beta(s) disclosed herein may be administered to the subject in a single bolus delivery, via continuous delivery (e.g., continuous transdermal, mucosal, or intravenous delivery) over an extended time period, or in a repeated administration protocol (e.g., by an hourly, daily or weekly, repeated administration protocol). In this context, a therapeutically effective dosage of the interferon beta(s) may include repeated doses within a prolonged prophylaxis or treatment regimen, that will yield clinically significant results to alleviate one or more symptoms or detectable conditions associated with a targeted disease or condition as set forth above. Determination of effective dosages in this context is typically based on animal model studies followed up by human clinical trials and is guided by determining effective dosages and administration protocols that significantly reduce the occurrence or severity of targeted disease symptoms or conditions in the subject. Suitable models in this regard include, for example, murine, rat, porcine, feline, non-human primate, and other accepted animal model subjects known in the art. Alternatively, effective dosages can be determined using in vitro models (e.g., immunologic and histopathologic assays). Using such models, only ordinary calculations and adjustments are typically required to determine an appropriate concentration and dose to administer a therapeutically effective amount of the interferon beta(s) (e.g., amounts that are intranasally effective, transdermally effective, intravenously effective, or intramuscularly effective to elicit a desired response). In alternative embodiments, an “effective amount” or “effective dose” of the interferon beta(s) may simply inhibit or enhance one or more selected biological activity(ies) correlated with a disease or condition, as set forth above, for either therapeutic or diagnostic purposes.

The actual dosage of interferon betas will of course vary according to factors such as the disease indication and particular status of the subject (e.g., the subject's age, size, fitness, extent of symptoms, susceptibility factors, etc), time and route of administration, other drugs or treatments being administered concurrently, as well as the specific pharmacology of the interferon beta(s) for eliciting the desired activity or biological response in the subject. Dosage regimens may be adjusted to provide an optimum prophylactic or therapeutic response. A therapeutically effective amount is also one in which any toxic or detrimental side effects of the interferon beta is outweighed in clinical terms by therapeutically beneficial effects. A non-limiting range for a therapeutically effective amount of a interferon beta within the methods and formulations of the invention is 0.01 μg/kg-10 mg/kg, more typically between about 0.05 and 5 mg/kg, and in certain embodiments between about 0.2 and 2 mg/kg. Dosages within this range can be achieved by single or multiple administrations, including, e.g., multiple administrations per day, daily or weekly administrations. Per administration, it is desirable to administer at least one microgram of the interferon beta (e.g., one or more interferon-β peptides, proteins, analogs and mimetics, and other interferon betas), more typically between about 10 μg and 5.0 mg, and in certain embodiments between about 100 μg and 1.0 or 2.0 mg to an average human subject. It is to be further noted that for each particular subject, specific dosage regimens should be evaluated and adjusted over time according to the individual need and professional judgment of the person administering or supervising the administration of the permeabilizing peptide(s) and other interferon beta(s).

Dosage of interferon betas may be varied by the attending clinician to maintain a desired concentration at the target site. For example, a selected local concentration of the interferon beta in the bloodstream or CNS may be about 1-50 nanomoles per liter, sometimes between about 1.0 nanomole per liter and 10, 15 or 25 nanomoles per liter, depending on the subject's status and projected or measured response. Higher or lower concentrations may be selected based on the mode of delivery, e.g., trans-epidermal, rectal, oral, or intranasal delivery versus intravenous or subcutaneous delivery. Dosage should also be adjusted based on the release rate of the administered formulation, e.g., of a nasal spray versus powder, sustained release oral versus injected particulate or transdermal delivery formulations, etc. To achieve the same serum concentration level, for example, slow-release particles with a release rate of 5 nanomolar (under standard conditions) would be administered at about twice the dosage of particles with a release rate of 10 nanomolar.

Additional guidance as to particular dosages for selected interferon betas for use within the invention may be found widely disseminated in the literature. This is true for many of the therapeutic peptide and protein agents disclosed herein.

Kits

The instant invention also includes kits, packages and multicontainer units containing the above described pharmaceutical compositions, active ingredients, and/or means for administering the same for use in the prevention and treatment of diseases and other conditions in mammalian subjects. Briefly, these kits include a container or formulation that contains one or more interferon-β peptides, proteins, analogs and mimetics, and other interferon betas disclosed herein formulated in a pharmaceutical preparation for mucosal delivery. The interferon beta(s) is/are optionally contained in a bulk dispensing container or unit or multi-unit dosage form. Optional dispensing means may be provided, for example a pulmonary or intranasal spray applicator. Packaging materials optionally include a label or instruction indicating that the pharmaceutical agent packaged therewith can be used mucosally, e.g., intranasally, for treating or preventing a specific disease or condition.

Aerosal Nasal Administration of Interferon Beta

We have discovered that the Interferon beta-binding peptides can be administered intranasally using a nasal spray or aerosol. This is surprising because many proteins and peptides have been shown to be sheared or denatured due to the mechanical forces generated by the actuator in producing the spray or aerosol. In this area the following definitions are useful.
1. Aerosol—A product that is packaged under pressure and contains therapeutically active ingredients that are released upon activation of an appropriate valve system.
2. Metered aerosol—A pressurized dosage form comprised of metered dose valves, which allow for the delivery of a uniform quantity of spray upon each activation.
3. Powder aerosol—A product that is packaged under pressure and contains therapeutically active ingredients in the form of a powder, which are released upon activation of an appropriate valve system.
4. Spray aerosol—An aerosol product that utilizes a compressed gas as the propellant to provide the force necessary to expet the product as a wet spray; it generally applicable to solutions of medicinal agents in aqueous solvents.
5. Spray—A liquid minutely divided as by a jet of air or steam. Nasal spray drug products contain therapeutically active ingredients dissolved or suspended in solutions or mixtures of excipients in nonpressurized dispensers.
6. Metered spray—A non-pressurized dosage form consisting of valves that allow the dispensing of a specified quantity of spray upon each activation.
7. Suspension spray—A liquid preparation containing solid particles dispersed in a liquid vehicle and in the form of course droplets or as finely divided solids.

The fluid dynamic characterization of the aerosol spray emitted by metered nasal spray pumps as a drug delivery device (“DDD”). Spray characterization is an integral part of the regulatory submissions necessary for Food and Drug Administration (“FDA”) approval of research and development, quality assurance and stability testing procedures for new and existing nasal spray pumps.

Thorough characterization of the spray's geometry has been found to be the best indicator of the overall performance of nasal spray pumps. In particular, measurements of the spray's divergence angle (plume geometry) as it exits the device; the spray's cross-sectional ellipticity, uniformity and particle/droplet distribution (spray pattern); and the time evolution of the developing spray have been found to be the most representative performance quantities in the characterization of a nasal spray pump. During quality assurance and stability testing, plume geometry and spray pattern measurements are key identifiers for verifying consistency and conformity with the approved data criteria for the nasal spray pumps.

Definitions

Plume Height—the measurement from the actuator tip to the point at which the plume angle becomes non-linear because of the breakdown of linear flow. Based on a visual examination of digital images, and to establish a measurement point for width that is consistent with the farthest measurement point of spray pattern, a height of 30 mm is defined for this study
Major Axis—the largest chord that can be drawn within the fitted spray pattern that crosses the COMw in base units (mm)
Minor Axis—the smallest chord that can be drawn within the fitted spray pattern that crosses the COMw in base units (mm)
Ellipticity Ratio—the ratio of the major axis to the minor axis
D₁₀—the diameter of droplet for which 10% of the total liquid volume of sample consists of droplets of a smaller diameter (μm)
D₅₀—the diameter of droplet for which 50% of the total liquid volume of sample consists of droplets of a smaller diameter (μm), also known as the mass median diameter
D₉₀—the diameter of droplet for which 90% of the total liquid volume of sample consists of droplets of a smaller diameter (μm)
Span—measurement of the width of the distribution, The smaller the value, the narrower the distribution. Span is calculated as

$$\frac{( {D}_{90} - {D}_{10} )}{{D}_{50}} .$$
% RSD—percent relative standard deviation, the standard deviation divided by the mean of the series and multiplied by 100, also known as % CV.

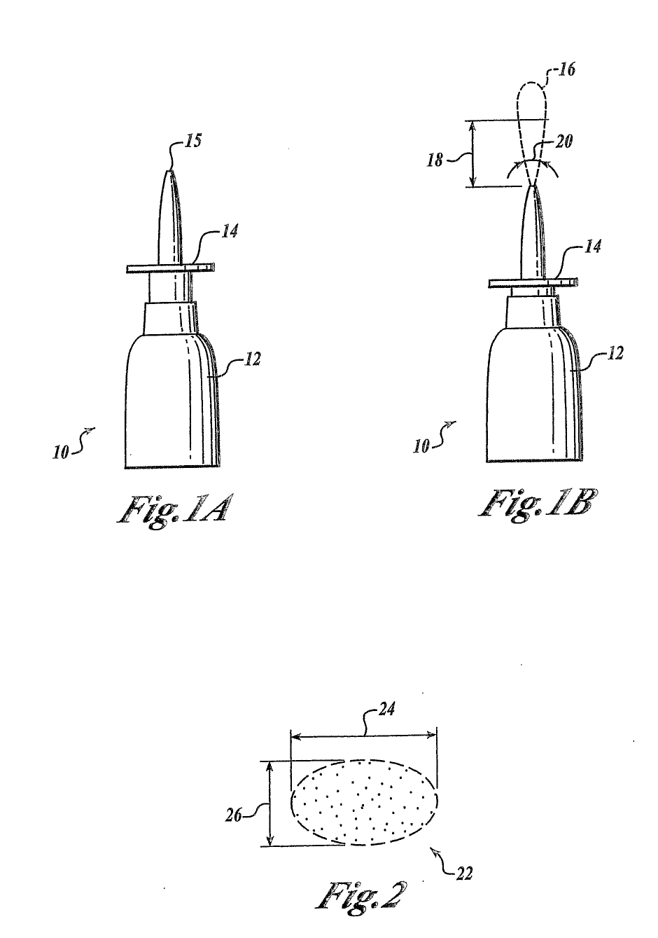

FIGS. 1A and 1B show a nasal spray device 10 before engagement (FIG. 1A) and after engagement (FIG. 1B). The nasal spray bottle 10 is comprised of a bottle 12 into which is the nasal Interferon beta-binding peptide formulation is placed, and an actuator 14, which when actuated or engage forces a spray plume, 16, of the Interferon beta-binding peptide out of the spray bottle, 12, through the actuator, 14. A spray pattern is determined by taking a photograph of a cross-section of the spray plume 16 above a predetermined height, 18, of the plume. The spray plume also has angle of ejection, 20, as it leaves actuator, 14. A spray pattern of spray plume 16 is shown on FIG. 2. Spray pattern 22, is elliptical and has a major axis, 24, and a minor axis 26.

The following examples are provided by way of illustration, not limitation.

### EXAMPLE 1

### Preparation of Intranasal Beta Interferon-β (IFN-β) Free of a Stablizer that is a Protein or Polypeptide

Four formulations of intranasal interferon beta-1a were prepared and tested in a permeation assay to determine the percentage permeation of interferon beta in each of the formulations listed below.

Formula 1 was comprised of an aqueous solution of interferon beta-1a (AVONEX®, Biogen, Cambridge, Mass.) having a concentration of interferon beta-la of 50 μg/mL, a pH of 4.8 and a calculated osmolarity of 250.

Formula 2 was comprised of an aqueous solution of interferon beta-1a (AVONEX®, Biogen, Cambridge, Mass.) having a concentration of interferon beta-1a of 50 μg/mL, 4.5 mg/mL of methyl-beta cyclodextrin, 1 mg/mL of EDTA, 1 mg/mL of DDPC, a pH of 4.8 and a calculated osmolarity of 300.

Formula 3 was comprised of an aqueous solution of interferon beta-1a (AVONEX®, Biogen, Cambridge, Mass.) having a concentration of interferon beta-1a of 50 μg/mL, 15 mg/mL of human serum albumin a pH of 4.8 and a calculated osmolarity of 250.

Formula 4 was comprised of an aqueous solution of interferon beta-1a (AVONEX®, Biogen, Cambridge, Mass.) having a concentration of interferon beta-1a of 50 μg/mL, 4.5 mg/mL of methyl-beta cyclodextrin, 1 mg/mL of EDTA, 1 mg/mL of DDPC, 15 mg/mL of human serum albumin, a pH of 4.8 and a calculated osmolarity of 300.

The procedures for determining the concentrations of interferon betas as test materials for evaluating enhanced permeation of active agents in conjunction with coordinate administration of mucosal delivery-enhancing agents or combinatorial formulation of the invention are generally as described above and in accordance with known methods and specific manufacturer instructions of ELISA kits employed for each particular assay. Permeation kinetics of the interferon beta is generally determined by taking measurements at multiple time points (for example 15 min., 30 min., 60 min. and 120 min) after the interferon beta is contacted with the apical epithelial cell surface (which may be simultaneous with, or subsequent to, exposure of the apical cell surface to the mucosal delivery-enhancing agent(s)).

EpiAirway™ tissue membranes are cultured in phenol red and hydrocortisone free medium (MatTek Corp., Ashland, Mass.). The tissue membranes are cultured at 37° C. for 48 hours to allow the tissues to equilibrate. Each tissue membrane is placed in an individual well of a 6-well plate containing 0.9 mL of serum free medium. 100 μL of the formulation (test sample or control) is applied to the apical surface of the membrane. Triplicate or quadruplicate samples of each test sample (mucosal delivery-enhancing agent in combination with a interferon beta, interferon-β) and control (interferon beta, interferon-β, alone) are evaluated in each assay. At each time point (15, 30, 60 and 120 minutes) the tissue membranes are moved to new wells containing fresh medium. The underlying 0.9 mL medium samples is harvested at each time point and stored at 4° C. for use in ELISA and lactate dehydrogenase (LDH) assays.

The ELISA kits are typically two-step sandwich ELISAs: the immunoreactive form of the agent being studied is first “captured” by an antibody immobilized on a 96-well microplate and after washing unbound material out of the wells, a “detection” antibody is allowed to react with the bound immunoreactive agent. This detection antibody is typically conjugated to an enzyme (most often horseradish peroxidase) and the amount of enzyme bound to the plate in immune complexes is then measured by assaying its activity with a chromogenic reagent. In addition to samples of supernatant medium collected at each of the time points in the permeation kinetics studies, appropriately diluted samples of the formulation (i.e., containing the subject biologically active test agent) that was applied to the apical surface of the units at the start of the kinetics study are also assayed in the ELISA plate, along with a set of manufacturer-provided standards. Each supernatant medium sample is generally assayed in duplicate wells by ELISA (it will be recalled that quadruplicate units are employed for each formulation in a permeation kinetics determination, generating a total of sixteen samples of supernatant medium collected over all four time points).

A. It is not uncommon for the apparent concentrations of active test agent in samples of supernatant medium or in diluted samples of material applied to the apical surface of the units to lie outside the range of concentrations of the standards after completion of an ELISA. No concentrations of material present in experimental samples are determined by extrapolation beyond the concentrations of the standards; rather, samples are rediluted appropriately to generate concentrations of the test material which can be more accurately determined by interpolation between the standards in a repeat ELISA.

B. The ELISA for a biologically active test agent, for example, interferon-β, is unique in its design and recommended protocol. Unlike most kits, the ELISA employs two monoclonal antibodies, one for capture and another, directed towards a nonoverlapping determinant for the biologically active test agent, e.g., interferon-β, as the detection antibody (this antibody is conjugated to horseradish peroxidase). As long as concentrations of IFN-β that lie below the upper limit of the assay are present in experimental samples, the assay protocol can be employed as per the manufacturer's instructions, which allow for incubation of the samples on the ELISA plate with both antibodies present simultanously. When the IFN-β levels in a sample are significantly higher than this upper limit, the levels of immunoreactive IFN-β may exceed the amounts of the antibodies in the incubation mixture, and some IFN-β which has no detection antibody bound will be captured on the plate, while some IFN-β which has detection antibody bound may not be captured. This leads to serious underestimation of the IFN-β levels in the sample (it will appear that the IFN-β levels in such a sample lie significantly below the upper limit of the assay). To eliminate this possibility, the assay protocol has been modified:

B.1. The diluted samples are first incubated on the ELISA plate containing the immobilized capture antibody for one hour in the absence of any detection antibody. After the one hour incubation, the wells are washed free of unbound material.

B.2. The detection antibody is incubated with the plate for one hour to permit formation of immune complexes with all captured antigen. The concentration of detection antibody is sufficient to react with the maximum level of IFN-β which has been bound by the capture antibody. The plate is then washed again to remove any unbound detection antibody.

B.3. The peroxidase substrate is added to the plate and incubated for fifteen minutes to allow color development to take place.

B.4. The “stop” solution is added to the plate, and the absorbance is read at 450 nm as well as 490 nm in the VMax microplate spectrophotometer. The absorbance of the colored product at 490 nm is much lower than that at 450 nm, but the absorbance at each wavelength is still proportional to concentration of product. The two readings ensure that the absorbance is linearly related to the amount of bound IFN-β over the working range of the VMax instrument (we routinely restrict the range from 0 to 2.5 OD, although the instrument is reported to be accurate over a range from 0 to 3.0 OD). The amount of IFN-β in the samples is determined by interpolation between the OD values obtained for the different standards included in the ELISA. Samples with OD readings outside the range obtained for the standards are rediluted and run in a repeat ELISA.

### RESULTS

Below are the percent permeation of the interferon beta for each of the formulations using two different ELISA assays, which detect the amount of interferon beta that permeate across the cellular barrier at a one-hour time point.

### Fujirebio Inc. ELISA Kit Results

| | Avg % perm |
|---|---|
| Formula 1 | 0.0033088 |
| Formula 2 | 0.531863 |
| Formula 3 | 0.0034314 |
| Formula 4 | 0.379902 |

### PBL Biomedical Lab

| | Avg % perm |
|---|---|
| Formula 1 | 0.01612485 |
| Formula 2 | 0.5267601 |
| Formula 3 | 0.007607575 |
| Formula 4 | 0.1359906 |

What is claimed is:

1. An intranasal interferon-β formulation comprised of interferon-β and a solubilizing agent wherein the formulation is free of a stabilizer that is protein or a polypeptide.

2. The formulation of claim 1 wherein the solubilizing agent is selected from the group consisting of cyclodextrin, α-cyclodextrin, hydroxypropyl-β-cyclodextran, sulfobutylether-β-cyclodextran, methyl-β-cyclodextrin and chitosan.

3. The formulation of claim 2 wherein the solubilizing agent is methyl-β-cyclodextran.

4. The formulation of claim 1 further comprised of a surface active agent.

5. The formulation of claim 4 wherein the surface active agent is selected from the group consisting of L-α-phosphatidylcholine didecanoyl (DDPC), polysorbate 20, polysorbate 80, polyethylene glycol (PEG), cetyl alcohol, polyvinylpyrolidone (PVP), polyvinyl alcohol (PVA), lanolin alcohol, sphingomyelin, phosphatidylethanolamine and sorbitan monooleate.

6. The formulation of claim 5 wherein the surface active agent is DDPC.

7. The formulation of claim 1 further comprised of a chelating agent.

8. The formulation of clam 6, wherein the chelating agent is selected from the group consisting of deferiprone, deferoxamine, ditiocarb sodium, penicillamine, pentetate calcium trisodium, pentetic acid, succimer, trientine, and EDTA.

9. The formulation of clam 6, wherein the chelating agent is EDTA.

10. The formulation of claim 3, further comprised of water.

11. The formulation of claim 10, wherein said formulation has a pH of from 3 to about 7.

12. The formulation of claim 11, wherein the pH is from 4 to about 5.

13. The formulation of claim 3, further comprising one or more sustained release-enhancing agent(s).

14. The formulation of claim 13, wherein the sustained release-enhancing agent is polyethylene glycol (PEG).

15. The formulation of claim 3, wherein said interferon-β is formulated in an effective dosage unit of between about 30 and 250 μg.

16. The formulation of claim 3, further comprising one or more steroid or corticosteroid compound(s), wherein said formulation is effective following mucosal administration to alleviate one or more symptom(s) of inflammation, nasal irritation, rhinitis, or allergy.

17. The formulation of claim 3, further comprising one or more steroid or corticosteroid compound(s), wherein said composition is effective following mucosal administration to alleviate one or more symptom(s) of an autoimmune disease or viral infection.

18. The formulation of claim 3, wherein permeability across an epithelial cell layer is enhanced 4- to 7-fold over a simple aqueous formulation comprising human serum albumin.

19. An aqueous interferon-β formulation for intranasal administration, comprised of interferon beta, DDPC, EDTA and methyl-β-cyclodextran, a pH between about 4 to about 5, wherein the formulation is free of a stabilizer that is protein or a polypeptide.

20. An intranasal interferon-β formulation comprised of interferon beta and a solubilizing agent, wherein the formulation is free of a stabilizer that is protein or a polypeptide, and wherein following nasal administration to a patient provides a interferon-β compound in blood plasma of the patient with a Tₘₐₓ between about 0.1 to about 1.0 hours.

21. The formulation of claim 20, wherein said formulation following mucosal administration to said patient yields a peak concentration of said human interferon-β compound(s) in said CNS tissue or fluid of the patient that is 10% or greater compared to a peak concentration of said human interferon-β compound(s) in a blood plasma of the patient.

22. The use of interferon-β in the manufacture of a medicament for treating an autoimmune or viral disease in a patient, comprising transmucosal administration of the medicament comprising an effective amount of interferon-β and a solubilizing agent, wherein the medicament is free of a stabilizer that is protein or a polypeptide.

23. The use of interferon-β of claim 22, wherein the administration is by intranasal delivery

24. The use of interferon-β of claim 23, wherein said medicament is provided in a multiple dosage unit kit or container for repeated self-dosing by said patient.

25. The use of interferon-β of claim 24, wherein said medicament is repeatedly administered through an intranasal effective dosage regimen that involves multiple administrations of said medicament to said patient during a daily or weekly schedule to maintain a therapeutically effective baseline level of interferon-β during an extended dosing period.

26. The use of interferon-β of claim 25, wherein said medicament is self-administered by said patient between two and six times daily to maintain a therapeutically effective baseline level of interferon-β during an 8 hour to 24 hour extended dosing period.

27. The use of interferon-β of claim 22, wherein said administration produces Cₘₐₓ of said interferon-β in a blood plasma or central nervous system tissue or fluid (CNS) of said patient following mucosal administration that is 25% or greater as compared to a peak concentration of interferon-β in blood plasma or CNS following intramuscular injection of an equivalent concentration or dose of interferon-β to said patient.

28. The use of interferon-β of claim 27, wherein said mucosal administration produces an area under concentration curve (AUC) of said interferon-β in a blood plasma or central nervous system tissue or fluid (CNS) that is 25% or greater compared intramuscular injection of an equivalent concentration or dose of interferon-β to said patient.

29. The use of interferon-β of claim 27, wherein mucosal administration of produces a Tₘₐₓ of said interferon-β in the blood plasma or CNS of the patient within about 0.1 to about 1.0 hours.

30. The use of interferon-β of claim 27, wherein said administration produces a peak concentration of said human interferon-β in the CNS of the patient that is 10% or greater compared to a peak concentration of said human interferon-β in the blood plasma of the patient.

31. The use of interferon-β of claim 22, wherein said medicament further comprises a plurality of mucosal delivery-enhancing agents.

32. The use of interferon-β of claim 31, wherein said medicament further comprises a sustained release-enhancing agents.

33. The use of interferon-β of claim 32, wherein the sustained release-enhancing agent is polyethylene glycol (PEG).

34. The use of interferon-β of claim 22, wherein said interferon-β is formulated in an effective dosage unit of between about 30 and 250 μg.

35. The use of interferon-β of claim 22, which is effective to alleviate one or more symptom(s) of chronic hepatitis B, condyloma acuminata, papillomavirus warts of the larynx or skin, or childhood viral encephalitis in said patient.

36. A pharmaceutical kit for nasal drug delivery comprising an aqueous formulation of interferon-β in a container, and a droplet-generating actuator attached to said container and fluidly connected to the solution in the container, wherein said formulation is substantially free of a stabilizer that is a protein or a polypeptide, wherein said actuator produces a spray of the formulation through a tip of the actuator when said actuator is engaged, and wherein said spray of solution has a spray pattern ellipticity ratio of from about 1.0 to about 1.4 when measured at a height of 3.0 cm from the actuator tip.

37. The kit of claim 36, wherein the spray is comprised of droplets of the formulation wherein less than 5% of the droplets are less than 10 μm in size.

38. The kit of claim 36, wherein the spray consists of a pattern with a major axis and minor axis of 25 and 40 mm, respectively.

39. The kit of claim 36, wherein the spray is comprised of droplets of the formulation wherein less than 50% of the droplets are 26.9 μm or less in size.

40. The kit of claim 36, wherein the spray is comprised of droplets of the formulation wherein 90% of the droplets are 55.3 μm or less in size.
